SEQUENCE LISTING (26 sequences)

SEQ ID NO: 1 - protein, 106 aa, Mus musculus
QIVLTQSPPIMSASLGEEITLTCSASSRVSYMLWYQQKSGTSPKLLIYSTSNLASGVPSRFSGSGSGTFYSLTISSVEAEDAADYYCHQWTSNPCTFGGGTKLEIK

SEQ ID NO: 2 - protein, 107 aa, Mus musculus
STVMTQTPKFLLVSAGDRVTITCKASQSVSNDVAWYQQKPGQSPKVLIYFASNRYTGVPDRFTGSGYGTDFTFTINTVQAEDLAVYFCQQDYSSPLTFGAGTKLELK

SEQ ID NO: 3 - protein, 106 aa, Mus musculus
QIVLSQSPAILSASPGEKVTMTCRASSSVSYMHWYQQRPGSSPKPWIYATSNLASGVPARFSGSGSGTSYYFTISRVEAEDAATYYCQQWNSNPPTHGGGTKLEIK

SEQ ID NO: 4 - protein, 106 aa, Mus musculus
QIVLSQSPAILSASPGEKVTMTCRASSSVSYMDWYQQKPGSSPKPWIYATSNLASGVPARFSGSGSGTSYSLTISRVEAEDTATYYCQQWTFNPPTFGSGTKLEIK

SEQ ID NO: 5 - protein, 106 aa, Mus musculus
QIVLSQSPAILSASPGEKVTMTCRASSSVSYMHWYQQKPGSSPKPWIYATSNLASGVPARFSGSGSGTSYSLTITRVEAEDAATYYCQQWTFNPPTFGAGTKLELK

SEQ ID NO: 6 - protein, 106 aa, Mus musculus
QIVLSQSPAILSSSPGEKVTMTCRASSSVSYMLWYQQKPGSSPEPWIYATSNLASGVPARFSGGGSGTSYSLTISRVEAEDAATYYCQQWTFNPPTFGGGTKLEIK

SEQ ID NO: 7 - protein, 107 aa, Mus musculus
DILLTQSPAILFVSPGERVSLSCRASQNIGTSIHWYQQRTNGSPRLLIKYASESFSGIPSRFSGSGSGTDFTLSINSVESEDIADYYCQQSNSWPFTFGSGTKLEIK

SEQ ID NO: 8 - protein, 108 aa, Mus musculus
EIILTQSPTTMAASPGEKITITCSASSSISSYYLRWYQQKPGFSPKVLIYRTSNLASGVPARFSGSGSGTSYSLTIGTMEAEDVATYYCQQGNTVPLTFGSGTKLEIK

SEQ ID NO: 9 - protein, 123 aa, Mus musculus
QVQLQQPGAELVKPGASVKMSCRASGYTFTNYNMHWIKQTPGQGLEWIGAIYPGSGDTSYNRKFKGKATLTADTSSSTAYMQFSSLTSADSAVYYCARFTYYYGGTYGAMDYWGQGTSVTVSL

SEQ ID NO: 10 - protein, 124 aa, Mus musculus
QIQLVQSGPELKKPGETVKISCKASGYTFTNFGVNWVKQAPGKGLKWMGWINTYTGEPSYADDFKGRFAFSLEASANTAYLQINNLKNDDMSTYFCTRRTNYYGTSYYYAMDYWGQGTSVTVSS

SEQ ID NO: 11 - protein, 119 aa, Mus musculus
QVQLQQPGAELVKPGASVKMSCKASGYTFTSYNIHWVKQTPGQGLEWIGAIYPGNGDTSYNQKFKGKATLTSDKSSSTAYMQLSSLTSEDSAVYYCARMSTMITGFDYWGQGTTLTVSS

SEQ ID NO: 12 - protein, 122 aa, Mus musculus
QVQLQQPGAELVKPGASVKMSCKASGFTFTSYNLHWVKQTPGQGLEWIGAIYPGSGDTSYNQQFKGKATLTADKSSNTAYMQLNSLTSEDSAVYCCARSAMISTGNWYFDYWGQGTTLTVSS

SEQ ID NO: 13 - protein, 121 aa, Mus musculus
QVQLQQPGAELVKPGASVKMSCKASGFTFTSYNMHWVKQTPGQGLEWIGGIYPGNGDTSYNQKFKGKATLTADKSSSTAYMQLSSLTSEDSAVYYCARFYYYGSMGAMDYWGQGTSVTVSS

SEQ ID NO: 14 - protein, 122 aa, Mus musculus
QVQLQQPGAELVKPGASVKMSCKASGYTFTTYNLHWVKQTPGQGLEWIGGIYPGNGDTSYNQKFKVKATLTADKSSNTAYMQLSSLTSEDSAVYYCARWIYYGNYEGTLDYWGQGTSVTVSS

SEQ ID NO: 15 - protein, 116 aa, Mus musculus
QVQLQQSGAELAKPGASVKMSCKASGYTFTSYWMHWVKQRPGQGLEWIGYITPSTGYTDYNKKFKDKATLTADRSSSTAYMHLSSLTSEDSAVYYCARSGPYFDVWGAGTTVTVSS

SEQ ID NO: 16 - protein, 119 aa, Mus musculus
QVQLQQPGAELVKPGASVKVSCKASGFTFTSYNLHWVKQTPGQGLVWIGAIYPGNGDTSYNQKFRGKATLTADISSSTAYMQLSSLTSEDSAVYYCARYYYGYDAMDYWGQGTSVTVSS

SEQ ID NO: 17 - protein, 107 aa, Artificial
  L chain V region sequence of humanized antibody abb 1791
STVMTQSPDSLAVSLGERATINCKSSQSVSNDVAWYQQKPGQSPKVLIYFASNRYSGVPDRFSGSGYGTDFTLTISSLQAEDVAVYFCQQDYSSPLTFGAGTKLEIK

SEQ ID NO: 18 - protein, 107 aa, Artificial
  L chain V region sequence of humanized antibody fra 1791
STVMTQSPSFLSASVGDRVTITCKASQSVSNDVAWYQQKPGQSPKVLIYFASNRYTGVPDRFSGSGYGTDFTLTISSLQAEDVAVYFCQQDYSSPLTFGAGTKLEIK

SEQ ID NO: 19 - protein, 107 aa, Artificial
  L chain V region sequence of humanized antibody sdr 1791
STVMTQSPDSLAVSLGERATINCKSSQSNSNDVAWYQQKPGQSPKVLIYFASNRYSGVPDRFSGSGYGTDFTLTISSLQAEDVAVYFCQQDYSSPLTFGAGTKLEIK

SEQ ID NO: 20 - protein, 107 aa, Artificial
  L chain V region sequence of humanized antibody Ven 1791
STVMTQSPDSLAVSLGERVTINCKASQSVSNDVAWYQQKPGQSPKVLIYFASNRYTGVPDRFSGSGYGTDFTFTISSVQAEDVAVYFCQQDYSSPLTFGAGTKLELK

SEQ ID NO: 21 - protein, 124 aa, Artificial
  H chain V region sequence of humanized antibody abb 1791
QIQLVQSGSELKKPGASVKVSCKASGYTFTNFGVNWVRQAPGKGLEWMGWINTYTGEPSYAQGFTGRFVFSLDASVSTAYLQISSLKAEDTATYFCTRRTNYYGTSYYYAMDYWGQGTTVTVSS

SEQ ID NO: 22 - protein, 124 aa, Artificial
  H chain V region sequence of humanized antibody fra 1791
QIQLVQSGSELKKPGASVKVSCKASGYTFTNFGVNWVKQAPGKGLKWMGWINTYTGEPSYADDFKGRFAFSLDASASTAYLQISSLKAEDMATYFCTRRTNYYGTSYYYAMDYWGQGTTVTVSS

SEQ ID NO: 23 - protein, 124 aa, Artificial
  H chain V region sequence of humanized antibody sdr 1791
QIQLVQSGSELKKPGASVKVSCKASGYTFTNFGVNWVRQAPGKGLKWMGWINTYTGEPSYAQGFTGRFAFSLDASVSTAYLQISSLKAEDTATYFCTRRTNYYGTSYYYAMDYWGQGTTVTVSS

SEQ ID NO: 24 - protein, 124 aa, Artificial
  H chain V region sequence of humanized antibody Ven 1791
QIQLVQSGPELKKPGASVKISCKASGYTFTNFGVNWVKQAPGKGLKWMGWINTYTGEPSYADDFKGRFAFSLDASVSTAYLQISSLKAEDTSTYFCTRRTNYYGTSYYYAMDYWGQGTTVTVSS

SEQ ID NO: 25 - DNA, 35 bp, Artificial sequence
  primer
aatgcggccgccaccatgacaacacccagaaattc

SEQ ID NO: 26 - DNA, 29 bp, Artificial sequence
  primer
gctctagattaaggagagctgtcattttc

(19) United States
(12) Patent Application Publication
(10) Pub. No.: US 2009/0203886 A1
(43) Pub. Date: Aug. 13, 2009

(54) HUMANIZED ANTI CD20 MONOCLONAL ANTIBODY

(75) Inventors: Susumu Uchiyama, Osaka (JP); Kiichi Fukui, Osaka (JP)

Correspondence Address: NIXON PEABODY, LLP, 401 9TH STREET, NW, SUITE 900, WASHINGTON, DC, 20004-2128, US

(73) Assignee: OSAKA UNIVERSITY, Suita-shi, Osaka (JP)

(21) Appl. No.: 12/281,805

(22) PCT Filed: Jul. 6, 2006
(86) PCT No.: PCT/JP2006/313499

§ 371 Date: Oct. 8, 2008

(30) Foreign Application Priority Data
Mar. 7, 2006 (JP) PCT/JP2006/304370

(51) Int. Cl. C07K 16/00

(52) U.S. Cl. 530/387.3

(57) ABSTRACT

The present invention provides a humanized anti human CD20 monoclonal antibody, selection criteria therefor, humanized antibodies selected using that criteria and showing biological characteristics suitable for use as pharmaceuticals.

### TECHNICAL FIELD

The present invention relates to an anti CD20 monoclonal antibody.

### BACKGROUND ART

CD20 is a protein not containing sugar chains, which is expressed on the cellular surface of human B lymphocytes. CD20 is expressed in the B cells of many malignant tumors in addition to expression in normal B cells in the peripheral blood, spleen, tonsils and bone marrow. Epitopes to which monoclonal antibodies of CD20 bind display extremely high variation and a wide variety of biological responses have been reported. Furthermore there have been many reports of monoclonal antibodies recognizing CD20. In particular, rituximab is a chimerized murine/human monoclonal antibody (C2B8) derived from a murine antibody 2B8 obtained by immunizing an SB cell strain, a type of human B cell (refer to Pamphlet of WO 94/11026; and U.S. Pat. No. 5,736,137). Rituximab is used under the name Rituxan® as a therapeutic agent for the treatment of low malignant non-Hodgkin's lymphoma (NHL). Subsequently it was reported that Rituxan is effective for many immune diseases related with B cells. For example, Rituxan has demonstrated efficacy against malignant tumors such as chronic lymphocytic leukemia (CCL), autoimmune diseases involving pathogenic autoantibodies such as autoimmune hemolyticanemia and idiopathic thrombocytopenic purpura (ITP), and inflammatory diseases such as rheumatoid arthritis (RA) and multiple sclerosis (refer to Coiffier B et al., Blood 1998; 92:1297-32; Edward J C et al., Rheumatology (Oxford) 2001; 40:205-11; Zaja F et al., Heamatologica 2002; 87:189-95; and Perrotta S et al., Br J Haematol 2002; 116:465-7).

It has been reported that human complement binds to rituximab conjugated to lymphatic B cells resulting in lysis of lymphatic B cell lines by complement-dependent cytotoxicity (CDC) (refer to Reff et al., Blood 1994; 83: 435-445). Rituximab has also displayed activity in assays for antibody-dependent cell-mediated cytotoxicity (ADCC) and induced apoptosis and growth inhibitory activity in tritiated thymidine incorporation assays (refer to Maloney et al., Blood 1996; 88: 637a).

Molecules chimerized from different animal types are antigenic and therefore are generally not desirable as therapeutic agents. However anti CD20 antibodies including rituximab are not antigenic since they target all B cells including normal cells followed by their deletion. However it has been reported that neutralizing antibodies, albeit several percent, are induced during therapy and that the level of dosage and therapeutic periods increase the probability of inducing neutralizing antibodies. In addition, there has been a shift in treatment targets from B cell lymphomas to RA, IT and MS. Therefore there is an increased focus on problems associated with antigenicity. Consequently recently there has been a need for human antibodies or humanized antibodies containing sequences close to human sequences.

Chimerized antibodies entail the problem that they have a relatively short half life in blood. The β half life (β½) of murine/human chimerized antibodies including rituximab is no more than 3 to 4 days. The efficacy in clinical trials of rituximab against low malignant NHL has been reported to be less than 50% (refer to IDEC Pharmaceuticals Corporation News Release, Dec. 8, 1998). Furthermore there is the problem that increases in dosages required in NHL therapy since the dissociation constant (Kd value) of rituximab for CD20 antibody is 5.2 nM and the corresponding binding affinity is not very high (refer to Mitchell E R et al., Blood 1994; 82:435-445).

### DISCLOSURE OF THE INVENTION

In view of the problems discussed above, the present invention has the principal object of providing an anti CD20 monoclonal antibody displaying biological activity suitable as a pharmaceutical.

In order to attain the above object, the present inventors performed diligent research into the preparation of a monoclonal antibody displaying high binding affinity to naturally-occurring human CD20 molecules in order to obtain an anti CD20 monoclonal antibody having excellent characteristics. As a result, the invention is based on the insight that a high-affinity monoclonal antibody displaying excellent biological activity is obtained by use of an immunogen such as SB cells or Raji cells which are B cell strains thought to contain a high density of CD20 antigen combined with a non-human animal cell modified using genetic recombination to express large amount of CD20 on the cellular membrane.

The present inventors succeeded in identifying a novel method of selecting effective anti human CD20 humanized antibodies. The use of this method of selection has enabled the selection of candidates from the humanized anti CD20 monoclonal antibodies of the present invention for use as effective therapeutic agents.

In other words, the present invention provides the followings:

(1) A humanized anti CD20 monoclonal antibody which has growth inhibiting activity on cells having human CD20 antigen, the immunogen being a human B cell strain expressing human CD20 antigen and a cell strain transformed with human CD20 DNA, which is a non-human cell and derived from an animal which is different from the animal to be immunized, and which meets the selection criteria below:

(i) the antibody having a dissociation constant (Kd value) for human CD20 antigen of less than approximately 9.5 nM and CDC activity on B cells equal to or greater than that of 2B8 antibody;

(2) A humanized anti CD20 monoclonal antibody which has growth inhibiting activity on cells containing human CD20 antigen, the immunogen being a human B cell strain expressing human CD20 antigen and a cell strain transformed with human CD20 DNA, which is a non-human cell and derived from an animal which is different from the animal to be immunized, and which meets the selection criteria below:

(a) the antibody having a dissociation constant (Kd value) for human CD20 antigen of less than approximately 9.5 nM and CDC activity on Raji cells (suspended cells) or SU-DHL4 cells equal to or greater than that of 2B8 antibody;

(3) A humanized anti CD20 monoclonal antibody which has growth inhibiting activity on cells containing human CD20 antigen, the immunogen being a human B cell strain expressing human CD20 antigen and a cell strain transformed with human CD20 DNA, which is a non-human cell and derived from an animal which is different from the animal to be immunized, and which meets the selection criteria below:

(ii) the antibody having a Kd value for human CD20 antigen in the range of from approximately 9.5 nM to approximately 13 nM and a total of apoptosis activity and CDC activity on B cells equal to or greater than that of 2B8 antibody;

(4) A humanized anti CD20 monoclonal antibody which has growth inhibiting activity on cells containing human CD20 antigen, the immunogen being a human B cell strain expressing human CD20 antigen and a cell strain transformed with human CD20 DNA which is a non-human cell and derived from an animal which is different from the animal to be immunized, and which meets the selection criteria below:

(b) the antibody having a Kd value for human CD20 antigen in the range of from approximately 9.5 nM to approximately 13 nM and a total of apoptosis activity and CDC activity on WiL2 cells or RCKS cells equal to or greater than that of 2B8 antibody;

(5) The humanized anti CD20 monoclonal antibody according to the above-described 1 or 2, comprising a combination of the L chain set forth in SEQ ID No: 18 and the H chain set forth in SEQ ID No: 22;
(6) The humanized anti CD20 monoclonal antibody according to the above-described (1) or (2), comprising a combination of the L chain set forth in SEQ ID No: 18 and the H chain set forth in SEQ ID No: 24;
(7) The humanized anti CD20 monoclonal antibody according to the above-described (3) or (4), comprising a combination of the L chain set forth in SEQ ID No: 19 and the H chain set forth in SEQ ID No: 22; and
(8) A therapeutic agent for the treatment of B cell mediated diseases, comprising as an active ingredient the humanized anti CD20 monoclonal antibody according to any one of the above-described (1) to (7).

The present invention provides a humanized anti CD20 monoclonal antibody displaying a high binding affinity against extracellular epitopes of CD20 antigen and high cytotoxic activity such as CDC. These antibodies are extremely effective as therapeutic agents for diseases related with B cells.

### BRIEF DESCRIPTION OF THE DRAWINGS

FIG. 1 is a restriction map showing the structure of a vector pNOW-Ab for expressing a transformed antibody.

FIG. 2 is a restriction map showing the structure of a vector pNOW for expressing protein.

FIG. 3a is a graph showing the results of an apoptosis test.

FIG. 3b is a graph showing the results of an apoptosis test.

FIG. 3c is a graph showing the results of an apoptosis test.

FIG. 3d is a graph showing the results of an apoptosis test.

FIG. 4a is a graph showing the relationship of antibody concentration and ADCC.

FIG. 4b is a graph showing the relationship of antibody concentration and ADCC.

FIG. 4c is a graph showing the relationship of antibody concentration and ADCC.

FIG. 4d is a graph showing the relationship of antibody concentration and ADCC.

FIG. 5a is a graph showing the relationship of E:T ratio and ADCC.

FIG. 5b is a graph showing the relationship of E:T ratio and ADCC.

FIG. 5c is a graph showing the relationship of E:T ratio and ADCC.

FIG. 5d is a graph showing the relationship of E:T ratio and ADCC.

FIG. 6a is a graph showing the results of a CDC test.

FIG. 6b is a graph showing the results of a CDC test.

FIG. 6c is a graph showing the results of a CDC test.

FIG. 6d is a graph showing the results of a CDC test.

FIG. 7a is a graph showing the results of an apoptosis test using a murine antibody.

FIG. 7b is a graph showing the results of an apoptosis test using a murine antibody.

FIG. 7c is a graph showing the results of an apoptosis test using a murine antibody.

FIG. 7d is a graph showing the results of an apoptosis test using a murine antibody.

FIG. 8a is a graph showing the results of an apoptosis test using a humanized antibody.

FIG. 8b is a graph showing the results of an apoptosis test using a humanized antibody.

FIG. 8c is a graph showing the results of an apoptosis test using a humanized antibody.

FIG. 8d is a graph showing the results of an apoptosis test using a humanized antibody.

FIG. 9a is a graph showing the ratio of early apoptosis test using a humanized antibody.

FIG. 9b is a graph showing the ratio of early apoptosis test using a humanized antibody.

FIG. 9c is a graph showing the ratio of early apoptosis test using a humanized antibody.

FIG. 9d is a graph showing the ratio of early apoptosis test using a humanized antibody.

FIG. 10a is a graph showing the relationship between dissociation constant of humanized antibody and cytotoxicity (apoptosis inducing activity and CDC activity) Cells used are Raji cells. The white square shows CDC activity (%), the black circle shows apoptosis activity (%).

FIG. 10b is a graph showing the relationship between dissociation constant of humanized antibody and cytotoxicity (apoptosis inducing activity and CDC activity). Cells used are SU-DHL4 cells. The white square shows CDC activity (%), the black circle shows apoptosis activity (%)

FIG. 10c is a graph showing the relationship between dissociation constant of humanized antibody and cytotoxicity (apoptosis inducing activity and CDC activity). Cells used are WiL2 cells. The white square shows CDC activity (%), the black circle shows apoptosis activity (%).

FIG. 10d is a graph showing the relationship between dissociation constant of humanized antibody and cytotoxicity (apoptosis inducing activity and CDC activity). Cells used are RC-K8 cells. The white square shows CDC activity (%), the black circle shows apoptosis activity (%).

FIG. 11a is a graph showing CDC activity of humanized antibody and chimerized antibody. Cells used are Raji cells.

FIG. 11b is a graph showing CDC activity of humanized antibody and chimerized antibody. Cells used are SU-DHL4 cells.

FIG. 11c is a graph showing CDC activity of humanized antibody and chimerized antibody. Cells used are WiL2 cells.

FIG. 11d is a graph showing CDC activity of humanized antibody and chimerized antibody. Cells used are RC-K8 cells.

FIG. 12a is a graph showing the relationship of humanized antibody concentration and ADCC. Cells used are Raji cells.

FIG. 12b is a graph showing the relationship of humanized antibody concentration and ADCC. Cells used are SU-DHL4 cells.

FIG. 12c is a graph showing the relationship of humanized antibody concentration and ADCC. Cells used are WiL2 cells.

FIG. 12d is a graph showing the relationship of humanized antibody concentration and ADCC. Cells used are RC-K8 cells.

FIG. 13a is a graph showing the relationship of the E:T ratio for humanized antibody and ADCC. Cells used are Raji cells.

FIG. 13b is a graph showing the relationship of the E:T ratio for humanized antibody and ADCC. Cells used are SU-DHL4 cells.

FIG. 13c is a graph showing the relationship of the E:T ratio for humanized antibody and ADCC. Cells used are WiL2 cells.

FIG. 13d is a graph showing the relationship of the E:T ratio for humanized antibody and ADCC. Cells used are RC-K8 cells.

### DESCRIPTION OF ABBREVIATIONS

Pcmv: Cytomegalovirus promoter
PAbgh: Signal with poly A for bovine growth hormone gene
Psvd: Promoter for simian virus 40 deficient in enhancer
DHFR: cDNA for murine dihydrofolic acid reductase
PAsv: Signal with poly A for simian virus 40
PBR322ori: Replication origin in E. coli
Amp^{r}: Selective marker in E. coli (ampicillin resistance)
Neo^{r}: Selective marker in mammalian cells (G418 resistance)
INrbg: Intron for rabbit β globin
SPl: Signal peptide for antibody light chain
VL: cDNA for antibody light chain variable region
Cκ: cDNA for antibody K light chain constant region
SPh: Signal peptide for antibody light chain
Vh: cDNA for antibody light chain variable region
Cγ1: cDNA for antibody γ1 heavy chain constant region

### BEST MODE FOR CARRYING OUT THE INVENTION

In this specification, “antibody” refers to not only a whole antibody but also a fragment displaying equivalent binding affinity to antigen as that of the whole antibody, and for example, includes fragments (Fab, F(ab′)₂) containing the variable region of the original whole antibody.

The monoclonal antibodies according to the present invention are monoclonal antibodies which display high affinity to human CD20 antigen and have excellent biological activity and include murine-derived monoclonal antibodies, in addition to chimerized and humanized variants thereof.

A monoclonal antibody according to a first preferred embodiment has growth inhibiting activity on cells expressing human CD20 antigen, has a dissociation constant (Kd value) for human CD20 antigen of less than ½ of 2B8, which is a murine antibody derived from Rituximab, and preferably a Kd value of 1.7 to 3.39 nM and a high affinity to human CD20 antigen.

There is no particular limitation on the method of measuring the dissociation constant (Kd value) as long as the method allows the measurement of a Kd value with respect to antigen presented on cells. However in this specification, the dissociation constant is taken to be obtained by the method described hereinafter in the Examples.

The growth inhibitory activity against cells expressing human CD20 antigen should preferably be greater than that of 2B8. The growth inhibitory activity is preferably growth inhibitory activity with respect to in vitro culturing of cells expressing human CD20 antigen cultured in the absence of peripheral blood monocytes. More preferably, the growth inhibitory activity induces apoptosis. It has been reported that the binding of some anti CD20 antibodies to CD20 increases the concentration of calcium ions (Ca²⁺) in B cells and thus induces apoptosis mediated by Src kinase.

The measurement of the growth inhibitory activity above may be performed using the method described in Miyamoto T, Min W, Lillehoj H S. Avian Dis. 2002 January-March; 46(1):10-6.

A specific example of a monoclonal antibody according to a first embodiment of the present invention is a murine-derived monoclonal antibody wherein the L chain variable region amino acid sequence and the H chain variable region amino acid sequence are respectively SEQ ID Nos: 1 and 9, SEQ ID Nos: 2 and 10, or SEQ ID Nos: 3 and 11 in addition to chimerized or humanized variants thereof.

A chimerized antibody may be produced by fusing a variable region amino acid sequence from a murine-derived monoclonal antibody with a human immunoglobin constant region amino acid sequence according to a known method such as described for example in Ishida T, Imai K, Nippon Rinsho Vol 60, No 3, 2002-3:439-444.

Humanizing may be performed for example using a variable region CDR amino acid sequence from a murine-derived monoclonal antibody and a human immunoglobin amino acid sequence according to a known method such as described in Ishida T, Imai K, Nippon Rinsho Vol 60, No 3, 2002-3:439-444, Eduardo A. Padlan, Molecular Immunology, Vol. 28-4/5, pp 489-498, 1991; Eduardo A. Padlan et. al., The PASEB Journal, vol. 9, pp 133-139; and Tai te Wu, Elvin A. Kabat, Molecular Immunology, Vol. 29-9, pp 1141-1146, 1992.

When producing chimerized or humanized antibodies, an amino acid sequence from an L chain variable region of a plurality of murine monoclonal antibodies may be combined in an arbitrary manner with amino acid sequences from the H chain variable region. Examples include a chimerized anti CD20 monoclonal antibody combining a chimerized H chain of a murine-derived monoclonal antibody variable region amino acid sequence set forth in SEQ ID Nos: 9 to 11 and a chimerized L chain of a murine-derived monoclonal antibody variable region amino acid sequence set forth in any one of SEQ ID Nos: 1 to 3 and a humanized anti CD20 monoclonal antibody combining a humanized H chain of a murine-derived monoclonal antibody variable region CDR sequence set forth in any one of SEQ ID Nos: 9 to 11 and a humanized L chain of a murine-derived monoclonal antibody variable region CDR amino acid sequence set forth in any one of SEQ ID Nos: 1 to 3.

An antibody according to a second preferred embodiment of the present invention is a murine-derived chimerized or humanized monoclonal antibody which has a dissociation constant (Kd value) less than or equal to ⅛ that of 2B8 for human CD20 antigen.

It is well known that when an antibody which has a high affinity for human CD20 antigen, and in particular an antibody containing human IgG1 or IgG3 or an engineered a human Fc sequence version thereof (including murine-derived chimerized or humanized monoclonal antibodies) binds to CD20 on the cellular membrane, effector-cell activity is induced via FcγRIII (CD16) on NK cells resulting in antibody-dependent cell-mediated cytotoxicity (ADCC). It is well known that when an antibody which has high affinity to humanized CD20 antigen, and in particular an antibody containing human IgG1 or IgG3 or an engineered a human Fc sequence version thereof (including murine-derived chimerized or humanized monoclonal antibodies) binds to CD20 on the cellular membrane, the antibody induces complement activity and causes complement-dependent cytotoxicity (CDC).

Therefore an antibody according to a second preferred embodiment of the present invention is expected to display ADCC or CDC.

A specific example of an antibody according to a second embodiment of the present invention an antibody wherein the L chain variable region amino acid sequence and the H chain variable region amino acid sequence are respectively SEQ ID Nos: 4 and 12, SEQ ID Nos: 5 and 13, SEQ ID Nos: 6 and 14, SEQ ID Nos: 7 and 15 or SEQ ID Nos: 8 and 16.

Chimerized or humanized antibodies can be produced by a similar method to the first embodiment. An amino acid sequence from an L chain variable region of a plurality of murine-derived monoclonal antibodies may be combined in an arbitrary manner with amino acid sequences from the H chain variable region. Examples include a chimerized anti CD20 monoclonal antibody combining a chimerized H chain of a murine-derived monoclonal antibody variable region amino acid sequence set forth in any one of SEQ ID Nos: 12 to 16 and a chimerized L chain of a murine-derived monoclonal antibody variable region amino acid sequence set forth in any one of SEQ ID Nos: 4 to 8, and a humanized anti CD20 monoclonal antibody combining a humanized H chain of a murine-derived monoclonal antibody variable region CDR sequence set forth in any one of SEQ ID Nos: 12 to 16 and a humanized L chain of a murine-derived monoclonal antibody variable region CDR amino acid sequence set forth in any one of SEQ ID Nos: 4 to 8.

An antibody according to a third preferred embodiment of the present invention is a group of humanized monoclonal antibody not limited by a specific dissociation constant (Kd value) with respect to 2B8 and includes humanized monoclonal antibodies effective against cells with respect to which rituximab is not effective.

Examples of such antibodies include a humanized anti CD20 monoclonal antibody combining an L chain set forth in SEQ ID No: 18 and an H chain set forth in SEQ ID No: 24, an L chain set forth in SEQ ID No: 18 and an H chain set forth in SEQ ID No: 22, an L chain set forth in SEQ ID No: 19 and an H chain set forth in SEQ ID No: 22 and an L chain set forth in SEQ ID No: 19 and an H chain set forth in SEQ ID No: 23.

Furthermore the present inventors have classified humanized anti CD20 monoclonal antibodies obtained by the present invention by type based on a relationship between antibody affinity to human CD20 antigen and CDC activity, and apoptosis inducing activity and have selected monoclonal antibodies for use as antibody pharmaceuticals based on that classification (refer to Example 4).

In other words, the inventors realized that high-affinity antibodies could not induce apoptosis independently and required cross linking by a secondary antibody in order to induce apoptosis. On the other hand, the inventors realized that low affinity antibodies could induce apoptosis in isolation. Furthermore high antibody affinity was often found to correlate with high CDC activity. As a result, although low affinity antibodies could induce apoptosis without the presence of a secondary antibody, there was a tendency for low CDC activity. Thus these observations resulted in the formation of two selection criteria for candidate antibodies effective as therapeutic agents:

An antibody which can not induce apoptosis independently but which displays extremely high affinity to human CD20 antigen and CDC-mediated anti-cancer effects, or

An antibody which can induce apoptosis independently and which has high affinity to human CD20 antigen in addition to being an antibody which displays anti-cancer effects due to apoptosis and CDC.

In the former selection criterion, it is preferred that antibodies are selected which have a high affinity to human CD20 antigen and which have high CDC activity (display an inverse correlation between Kd value and CDC activity). Although clones satisfying the former selection criterion do not induce apoptosis independently, such clones have the advantage of high affinity and thus it is possible to promote cell deletion by CDC activity. The present inventors made the surprising discovery that a majority of antibodies satisfying the former selection criterion have a dissociation constant (Kd value) for human CD20 antigen of less than approximately 9.5 nM.

In the latter selection criterion, it is preferred that antibodies are selected which have a large total of CDC and apoptosis activity and which have a high affinity to human CD20 antigen. Although clones satisfying the latter selection criterion do not display high affinity, such clones have the advantage of high apoptosis activity and thus it is possible to promote cell deletion by a synergistic effect of CDC and inducing apoptosis. The present inventors made the surprising discovery that a majority of antibodies satisfying the latter selection criterion have a dissociation constant (Kd value) for human CD20 antigen in the range from approximately 9.5 nM to approximately 13 nM.

It is preferred that the selection criteria are expressed numerically. Thus it is possible to express the selection criteria in as follows:

(i) An antibody having a dissociation constant (Kd value) for human CD20 antigen of less than approximately 9.5 nM and CDC activity against B cells of equal to or greater than 2B8 antibody; or

(ii) An antibody having a Kd value for human CD20 antigen in the range of from approximately 9.5 nM to approximately 13 nM and a total of CDC activity and apoptosis activity against B cells equal to or greater than 2B8 antibody.

Herein, “equal” refers to a value in a range of approximately ±10% of the figure under comparison.

Experiments performed by the present inventors lead to the surprising result that it is preferred to apply the selection criteria according to the type of B cell. More precisely, in view of the results obtained in Example 4 described hereinafter, it is possible to define selection criterion (i) in greater detail in (a), and selection criteria (ii) in (b).

(a) An antibody having a dissociation constant (Kd value) for human CD20 antigen of less than approximately 9.5 nM and CDC activity against Raji cells (floating cells) or SU-DHL4 cells of equal to or greater than 2B8 antibody; or

(b) An antibody having a Kd value for human CD20 antigen in the range of from approximately 9.5 nM to approximately 13 nM and a total of CDC activity and apoptosis activity against WiL2 cells or RCK8 cells equal to or greater than 2B8 antibody.

Herein, “equal” refers to a value in a range of approximately 110% of the figure under comparison.

When applying the selection criteria in (i) or (a) above, it is preferred to select antibodies which have a Kd value for human CD20 antigen of less than approximately 9.5 nM, which is as low a Kd value as possible, and which have high CDC activity. Preferably, antibodies are selected which have CDC activity against Raji cells (floating cells) or DHL4 cells which is equal to or greater than 2B8 antibody. Normally there is a tendency that antibody with lower Kd values has higher antibody CDC activity, it is possible to simply select antibodies with small Kd values (high affinity to human CD20 antigen). Since antibodies satisfying selection criteria (i) or (a) have extremely high affinity to CD20 antigen as well as extremely high CDC activity, such antibodies display excellent anti-cancer effects. However antibodies satisfying selection criteria (i) or (a) can not induce apoptosis independently and require a secondary antibody in order to induce apoptosis.

When applying the selection criteria in (ii) or (b) above, it is preferred to select antibodies which have a small Kd value for human CD20 antigen in the range of approximately 9.5 nM to approximately 13 nM and which display the highest possible total of apoptosis activity and CDC activity. Preferably, antibodies are selected which have a total of apoptosis activity and CDC activity against WiL2 cells or RCK8 cells and the total is equal to or greater than 2B8 antibody. Since antibodies satisfying selection criteria (ii) or (b) have moderately high affinity to CD20 antigen and can induce apoptosis independently, such antibodies can display excellent anti-cancer effects as a result of the synergistic effect between apoptosis and CDC. Preferably, antibodies are selected which have a total of CDC activity and apoptosis activity equal to or greater than 258 antibody.

In other words, selection criteria (i) or (a) can be applied when it is expected that the antibody will have extremely high CDC activity. Selection criteria (ii) or (b) can be applied when it is expected that the antibody will have both CDC activity and apoptosis activity.

An example of a humanized CD20 monoclonal antibody obtained by the present invention which satisfies selection criteria (i) or (a) is a humanized CD20 monoclonal antibody combining an L chain set forth in SEQ ID No: 18 and an H chain set forth in SEQ ID No: 22 and a humanized CD20 monoclonal antibody combining an L chain set forth in SEQ ID No: 18 and an H chain set forth in SEQ ID No: 24.

An example of a humanized CD20 monoclonal antibody obtained by the present invention which satisfies selection criteria (ii) or (b) is a humanized CD20 monoclonal antibody combining an L chain set forth in SEQ ID No: 19 and an H chain set forth in SEQ ID No: 22.

It is possible to apply the selection process and methodology related to selection according to the present invention to antibodies recognizing epitopes which are different from the antibodies of the present invention, for example, antibodies obtained by a method other than that of the present invention. Thus the invention in a further embodiment relates to a method of selection of anticancer antibodies and antibodies selected by use of the method, the antibodies characterized by the following selection criteria:

An antibody which can not induce apoptosis independently but which displays extremely high affinity to antigen and CDC-mediated anti-cancer effects, or

An antibody which can induce apoptosis independently and which has high affinity to antigen in addition to being an antibody which displays anti-cancer effects due to apoptosis and CDC.

When applying selection criteria (i) or (a), or (ii) or (b), the measurement of affinity to antigen, CDC activity or apoptosis activity may be performed using any known method in the relevant field. Measurement of affinity to antigen is generally performed by using the measured the dissociation constant with respect to antigen as a standard. Measurement of the dissociation constant of humanized antibody with respect to human CD20 antibody is generally performed using cells expressing human CD20 antigen as a standard. It is preferred to use cells not expressing human CD20 antigen as a control. Methods such as attaching a detectable label to the humanized antibody, or using a labeled antibody specific to a human antibody can be used to detect humanized antibody binding to cells. For example, measurement of affinity to CD20 antigen (or dissociation constant, Kd values) can be performed as described in Example 2 hereinafter.

Isolation and selection of humanized anti CD20 antibodies according to the present invention will be described hereinafter.

A murine-derived monoclonal antibody which can be used in the preparation of a humanized anti CD20 antibody according to the present invention can be prepared by selecting a clone producing a monoclonal antibody having target characteristics from hybridoma clones produced by screening with the methods described hereinafter.

Sensitizing antigen (immunogen) can be obtained from SB cells or Raji cells which are cells expressing CD20 and, for example, CHO cells (CHO/CD20) expressing CD20 on the cellular membrane transformed by recombinant techniques using commercially available CD20 DNA (or filaments having the same effect). During initial immunization, additional immunization(s) and final immunization, immunization should be performed at least once during initial immunization and additional immunization(s) using either a cell strain which presents the sensitizing antigen and is derived from an animal of a different order from the animal being immunized or using a cell strain which presents the sensitizing antigen on the cellular surface membrane as a result of genetic recombination and is derived from an animal of the same order as the animal being immunized. The other cell strain is used during final immunization.

Other conditions may be the same as normal conditions for methods of preparing hybridomas producing monoclonal antibodies. Hybridomas producing monoclonal antibodies are prepared by known methods such as (1) immunizing an animals to be immunized (2) preparation of lymphocytes from immunized animals, (3) preparation of parent cells, (4) cell fusion of lymphocytes and parent cells and (5) screening and cloning (see Monokuronaru kotai, Seikagaku Jikkenho (Monoclonal Antibody, Biochemical Experiment Method), edited by Ailsa M. Campbell, and translated by Toshiaki OSAWA, Tokyo Kagaku Dojin (1989).

Methods of preparing monoclonal antibodies using cloned hybridomas may employ hybridomas prepared using the method of hybridoma production according to the present invention or may use a widely employed method of preparing monoclonal antibodies. Large-scale production can be effected for example by methods of cell culture or methods of producing murine ascites. Production of chimerized or humanized antibodies can be performed by producing genes coding for the chimerized or humanized antibody, transforming an expression vectors with the genes and expressing the expression vector in a suitable cell.

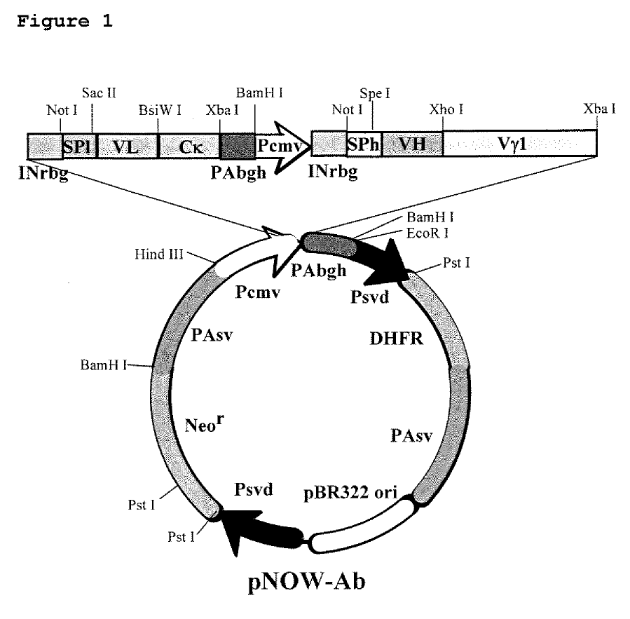

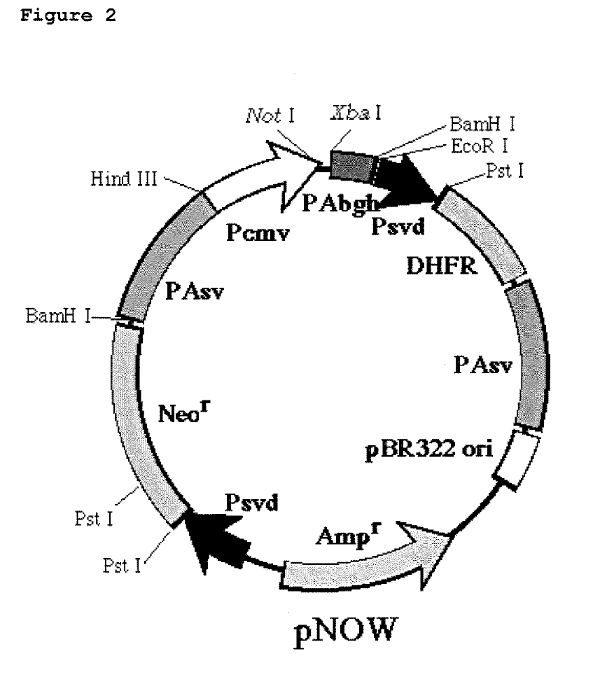

For example, variable region genes for the L chain and the H chain can be chimerized using constant regions genes for human immunoglobin L chain and H chain (κ) and combined with a CHO cell high expression vector. Although a commercially available vector system for production of recombinant antibodies can be used, it is possible to use a dimmer high expression vector pNOW-ab containing a multicloning site (MCS) for both L chains and H chains which is based on a high expression vector for mammalian cells (Japanese Patent No. 3,582,965). Restriction maps showing the structure of the vectors are shown in FIGS. 1 and 2. The expression vectors containing chimerized antibody genes are used to transfect CHO cells and then highly-productive clones are isolated. Antibodies are produced from these clones using known methods.

An antibody according to a first embodiment of the present invention displays relative high binding affinity compared to rituximab and growth inhibition activity. Preferably, since the antibody displays high activity in inducing apoptosis, a chimerized or humanized antibody can be used as an active ingredient of a therapeutic agent against diseases involving B cells and against B cell malignant tumors. Furthermore antibodies according to the second and third embodiments of the present invention are thought to display complement-dependent cytotoxicity (CDC) and antibody-dependent cell-mediated cytotoxicity (ADCC) against cells expressing human CD20 antigen and thus can be used as an active ingredient of a therapeutic agent against immune diseases involving B cells and against B cell malignant tumors. Thus the present invention also provides a therapeutic agent against diseases involving B cells which has as an active ingredient the chimerized or humanized antibodies of the invention.

The humanized anti CD20 monoclonal antibody according to the present invention can be selected using the selection criteria in (i) or (a), or in (ii) or (b) above. Antibodies satisfying these criteria are highly effective against immune diseases involving B cells and B cell malignant tumors and are particularly suitable for pharmaceutical use. Thus the invention provides a therapeutic agent for B cell mediated diseases containing, as an active ingredient, a humanized anti CD20 monoclonal antibody satisfying selection conditions (i) or (a), or (ii) or (b) above.

Two or more types of antibodies according to the present invention can be combined.

Diseases involving B cells are not limited to the following and include for example non-Hodgkin's lymphoma, chronic lymphocytic leukemia, acute lymphocytic leukemia, rheumatoid arthritis, autoimmune hemolyticanemia, idiopathic thrombocytopenic purpura, systemic lupus erythematosus, anti-phospholipid antibody syndrome, xerodermosteosis, Crohn's disease, chorionitis, and multiple sclerosis.

The therapeutic agent can be produced by known techniques and there is not particular limitation on processing ingredients. Dosages and the like can be determined by reference to known dosages for Rituxan.

The present invention will be described in further detail hereinafter with reference to the Examples. However the invention is not limited to the Examples.

### Example 1

### (1) Preparation of Immunogen for Immunosensitizing Mice

SB cells and Raji cells which are B cells strains expressing CD10 were cultured in vitro.

Separately thereto, DNA coding for the entire CD20 molecule (Multiple Choice cDNA human spleen, Origene Technologies, Inc. 6 Taft Court, Suite 100, Rockville, Md. 20850) was cloned using specific primers hCD20-S-GK-Not aatgcggccgccaccatgacaacacccagaaattc (SEQ ID No: 25) and hCD20-E-Xba gctctagattaaggagagctgtcattttc (SEQ ID No: 26). The DNA was inserted into a pNOW high expression vector for mammalian cells (FIG. 1) and used to transform CHO cells. Recombinant CHO cells (CD20/CHO cells) displaying high level of CD20 expression on the cellular surface were identified using FACS analysis. Staining was performed using FITC-labeled CD20 monoclonal antibodies and cells were selected as high expression cells when expressing five times or more the fluorescent intensity of SB cells.

### (2) Preparation of Immunogen

SB cells and Raji cells were cultured using RPM1640 medium supplemented with 10% FCS. CD20/CHO cells were cultured using CHO-S-SFMTI medium (Gibco, Cat. No. 12052-098) supplemented with 800 μg/ml of G418. These culture mediums were centrifuged for 5 minutes at 1,100 rpm, the cells were suspended in Dulbecco's PBS(−) and centrifuged again. This washing step was performed again, physiological salt solution was added to the cells and the prepared suspension (number of cells: 1-3×10⁷/ml) used for immunization.

### (3) Immunization

Both immunogen preparations were administered intraperitoneally to a 7-11 week female Balb/c mouse. After administering either SB cells or CD20/CHO cells on 2-3 occasions at various daily intervals, final immunization was performed using another cell type (CD20/CHO cells or Raji cells). The number of cells administered was 1-3×10⁷ cells per mouse for any type of cell.

The combinations of immunogens are shown in Table 1.

### (4) Cell Fusion

Three days after final immunization, spleen cells were recovered from two mice and fused with murine myeloma (NS-1) in the presence of PEG-1500 using the method described in Oi, V. T. and L. A. Herzenberg, 1980, in: Selected Methods in Cellular ˜Immunology, eds. B. Mishell and S. M. Shilgi (Freeman and Co. San Francisco, Calif.) p. 351.

### (5) Primary and Secondary Screening

A Cell ELISA assay was performed using a 96-wellplate having CD20/CHO cells or CHO cells (parent strain) attached thereto and wells producing antibodies reacting specifically to CD20 were selected. The same 96-wellplate with CD20/CHO cells attached thereto was used to perform a competitive reaction with rituximab (C2B8). Antibodies (wells) were selected which reacted to epitopes similar to C2B8.

The results of the screening are shown in Table 1.

### (6) Cell ELISA

CD20/CHO cells or CHO cells (parent strain) attached to a Poly-L-Lysine coated 96-wellplate (Asahi Techoglass Corporation, Cat. No. 11-023-018) were used in a cell ELISA assay. 150 μl of blocking buffer (PBS solution with 0.2% gelatin, 0.5% BSA) was introduced into each well and the plate was allowed to stand at 37° C. for one hour. The plate was washed five times using an aqueous solution of 150 nM-NaCl, 0.05%-Tween20 and then a 100 μl sample (a diluted solution of the culture supernatant) was introduced into each well. The primary reaction was conducted at 37° C. for one hour. After washing, 100 μl of a diluted solution of a labeled antibody (HRP-labeled anti murine IgG (H+L) rabbit antibody (Jackson Lab. Code No. 315-035-003) or HRP-labeled anti murine IgG (Fcγ) rabbit antibody (Jackson Lab. Code No. 315-035-008)) was introduced into each well and a secondary reaction was conducted at 37° C. for one hour. The same blocking solution was used in the preparation of the reaction solution for the primary and the secondary reactions. After washing, 100 μl of a color development solution (OPD) was introduced into each well and after 30 minutes 50 μl of 4N—H₂SO₄ was added to stop the reaction. The absorbance was measured at 492 nm.

### (7) Competitive Reaction in Cell ELISA

A mixed solution of a sample (diluted solution of culture supernatant) and chimerized antibody (10 to 40 ng/ml) was prepared.

After performing a blocking reaction as described above with respect to the Cell ELISA assay, 100 μl of the mixed solution was introduced into each well and the primary reaction was performed at 37° C. for one hour. After washing, 100 μl of a diluted solution of a labeled antibody (HRP-labeled anti human IgG (H+L) rabbit antibody (Jackson Lab. Code No. 309-035-082)) was introduced into each well and a secondary reaction was conducted at 37° C. for one hour. After washing, 100 μl of a color development solution (OPD) was introduced into each well and after 30 minutes 50 μl of 4N—H₂SO₄ was added to stop the reaction. The absorbance was measured at 492 nm.

Since the labeled antibody only reacts with the chimerized antibody, a reduction in the measured value should result from competition between the antibodies in the sample added in the primary reaction and the chimerized antibodies.

### (8) Cloning

A limiting dilution method was used. After culturing cells dispersed on a 96 wellplate, a Cell ELISA assay was performed on the culture supernatant of a well having a single colony in order to select clones producing specific antibodies.

### (9) Preparation of Purified Antibody

Clones producing specific antibodies were cultured in RPMI1640 medium supplemented with 10% FCS. When the cell density reached a value of approximately 5×10⁵/ml, the medium was replaced by serum-free culture medium ASF-104N (Ajinomoto). After 2 to 4 days, the culture solution was centrifuged, the culture supernatant recovered and a protein G column used to purify the antibody. The monoclonal antibody elution was dialyzed using 150 mM-NaCl. Filtration sterility was performed using a 0.2 μm filter in order to obtain the test antibody (anti human murine monoclonal antibody).

TABLE 1

| | | | | Primary, Secondary Screening | | |
|---|---|---|---|---|---|---|
| | Immunizing Method | | | Specificity against CD20 on | | |
| | Initial, | | | CD20/CHO cells | | |
| Cell | additional | | Immunizing | Selected Well | | Measured |
| Fusion | Immunizing, | Final | number of | No. | | Well |
| Series | times | immunizing | mouse | A | B | Number |
| 1K18 | SB cells, 3 times | Raji cells | 2 | 7 | 2 | 576 |
| 1K20 | Raji cells 3 times | SB cells | 2 | 7 | 0 | 576 |
| 1K14 | SB cells 2 times | CD20/CHO | 1 | 20 | 9 | 576 |
| | | cells | | | | |
| | SB cells 3 times | CD20/CHO | 1 | | | |
| | | cells | | | | |
| 1K17 | CD20/CHO cells 2 | Raji cells | 1 | 21 | >10 | 576 |
| | times | | | | | |
| | CD20/CHO cells 3 | Raji cells | 1 | | | |
| | times | | | | | |

Selected well number-A: well reacting with CD20/CHO cells and producing antibodies not reacting with CHO cells
Selected well number-B: of the wells selected in A, well producing antibodies undergoing a competitive reaction with the reference antibody (C2B8)

The L chain variable region amino acid sequence (SEQ ID Nos: 1 to 8) and the H chain variable region amino acid sequence (SEQ ID Nos: 9 to 16) of a representative monoclonal antibody producing 8 clones is shown below.

Amino acid sequence of H chain variable region of
1K0924 (SEQ ID No: 11):
QVQLQQPGAELVKPGASVKMSCKASGYTFTSYNIHWVKQTPGQGLEWIGA
IYPGNGDTSYNQKFKGKATLTSDKSSSTAYMQLSSLTSEDSAVYYCARMS
TMITGFDYWGQGTTLTVSS
Amino acid sequence of H chain variable region of
1K1228 (SEQ ID No: 16):
QVQLQQPGAELVKPGASVKVSCKASGFTFTSYNLHWVKQTPGQGLVWIGA
IYPGNGDTSYNQKFRGKATLTADISSSTAYMQLSSLTSEDSAVYYCARYY
YGYDAMDYWGQGTSVTVSS
Amino acid sequence of H chain variable region of
1K1422 (SEQ ID No: 9):
QVQLQQPGAELVKPGASVKMSCRASGYTFTNYNMHWIKQTPGQGLEWIGA
IYPGSGDTSYNRKFKGKATLTADTSSSTAYMQFSSLTSADSAVYYCARFT
YYYGGTYGAMDYWGQGTSVTVSL
Amino acid sequence of H chain variable region of
1K1791 (SEQ ID No: 10):
QIQLVQSGPELKKPGETVKISCKASGYTFTNFGVNWVKQAPGKGLKWMGW
INTYTGEPSYADDFKGRFAFSLEASANTAYLQINNLKNDDMSTYFCTRRT
NYYGTSYYYAMDYWGQGTSVTVSS
Amino acid sequence of H chain variable region of
1K1712 (SEQ ID No: 12):
QVQLQQPGAELVKPGASVKMSCKASGFTFTSYNLHWVKQTPGQGLEWIGA
IYPGSGDTSYNQQFKGKATLTADKSSNTAYMQLNSLTSEDSAVYCCARSA
MISTGNWYFDYWGQGTTLTVSS
Amino acid sequence of H chain variable region of
1K1402 (SEQ ID No: 13):
QVQLQQPGAELVKPGASVKMSCKASGFTFTSYNMHWVKQTPGQGLEWIGG
IYPGNGDTSYNQKFKGKATLTADKSSSTAYMQLSSLTSEDSAVYYCARFY
YYGSMGAMDYWGQGTSVTVSS
Amino acid sequence of H chain variable region of
1K1736 (SEQ ID No: 14):
QVQLQQPGAELVKPGASVKMSCKASGYTFTTYNLHWVKQTPGQGLEWIGG
IYPGNGDTSYNQKFKVKATLTADKSSNTAYMQLSSLTSEDSAVYYCARWI
YYGNYEGTLDYWGQGTSVTVSS
Amino acid sequence of H chain variable region of
1K1782 (SEQ ID No: 15):
QVQLQQSGAELAKPGASVKMSCKASSYTFTSYWMHWVKQRPGQCLEWICY
ITPSTGYTDYNKKFKDKATLTADRSSSTAYMHLSSLTSEDSAVYYCARSG
PYFDVWGAGTTVTVSS
Amino acid sequence of H chain variable region of
1K0924 (SEQ ID No: 3):
QIVLSQSPAILSASPGEKVTMTCRASSSVSYMHWYQQRPGSSPKPWIYAT
SNLASGVPARFSGSGSGTSYYFTISRVEAEDAATYYCQQWNSNPPTHGGG
TKLEIK
Amino acid sequence of H chain variable region of
1K1228 (SEQ ID No: 8):
EIILTQSPTTMAASPGEKITITCSASSSISSYYLRWYQQKPGFSPKVLIY
RTSNLASGVPARFSGSGSGTSYSLTIGTMEAEDVATYYCQQGNTVPLTFG
SGTKLEIK
Amino acid sequence of L chain variable region of
1K1422 (SEQ ID No: 1):
QIVLTQSPPIMSASLGEEITLTCSASSRVSYMLWYQQKSCTSPKLLIYST
SNLASGVPSRFSGSGSGTFYSLTISSVEAEDAADYYCHQWTSNPCTFGGS
TKLEIK
Amino acid sequence of L chain variable region of
1K1791 (SEQ ID No: 2):
STVMTQTPKFLLVSAGDRVTITCKASQSVSNDVAWYQQKPGQSPKVLIYF
ASNRYTGVPDRFTGSCYGTDETFTINTVQAEDLAVYFGQQDYSSPLTFGA
GTKLELK
Amino acid sequence of L chain variable region of
1K1712 (SEQ ID No: 4):
QIVLSQSPAILSASPGEKVTMTCRASSSVSYMDWYQQKPGSSPKPWIYAT
SNLASGVPARFSGSGSGTSYSLTISRVEAEDTATYYCQQWTFNPPTFGSG
TKLEIK
Amino acd sequence of L chain variable region of
1K1402 (SEQ ID No: 5):
QIVLSQSPAILSASPGEKVTMTCRASSSVSYMHWYQQKPGSSPKPWIYAT
SNLASGVPARFSGSGSGTSYSLTITRVEAEDAATYYCQQWTFNPPTFGAG
PKLELK
Amino acid sequence of L chain variable region of
1K1736 (SEQ ID No: 6):
QIVLSQSPAILSSSPCEKVTMTCRASSSVSYMLWYQQKFCSSPEPWIYAT
SNLASGVPARFSGGGSGTSYSLTISRVEAEDAATYYCQQWTFNPPTFGGG
TKLEIK
Amino acid sequence of L chain variable region of
1K1782 (SEQ ID No: 7):
DILLTQSPAILFVSPGERVSLSCRASQNIGTSIHWYQQRPNCSPRLLIKY
ASESFSGIPSRFSGSGSGTDFTLSTNSVESEDTADYYCQQSNSWPFTFGS
GTKLEIK

### Example 2

The base sequence of the variable region of the monoclonal antibody gene was determined for a portion of the resulting clones. Measurements of the antibody binding affinity and biological characteristic tests for monoclonals producing the sequences are described hereinafter.

### (1) Binding Affinity Measurement

Floating Raji cells derived from human B cells expressing the target antigen on the cellular surface and floating Jurkat cells derived from human T cells not expressing CD20 antigen were used. The cells were cultured in a CD20 incubator (SANYO MCO-175M) at 37° C. in an atmosphere of 5% CO₂ using RPMI1640 medium (nacalai tesque Co., Ltd., Cat. No. 30264-85, Lot L4K2844) supplemented with 10% fetal calf serum (FCS) (BIOLOGICAL IND. Cat. No. 04-001-1A, Lot 815242, a preparation which is preheated at 56° C. for 30 minutes in order to deactivate the complement component). The cells were maintained by subculturing twice per week.

Measurement of cell numbers was performed using a Burker-Turk hemacytometer (Erma, Inc., Cat. No. 03-303-1).

Confluent-cell culture medium three to four days after subculturing was centrifuged for three minutes at 3000 rpm at room temperature using a multipurpose refrigerated centrifuge LX-120 (TOMY Co. Ltd.). The supernatant was removed and the cells were recovered. The rotation speed and time used in this step were selected so that the number of cells displayed no change irrespective of the repetition of centrifugal separation and removal of the supernatant. In order to remove culturing medium and FCS remaining on the cell surface (washing), the recovered cells were suspended in Dulbecco's Phosphate Buffered Saline (−) (free of Ca and Mg, PBS (−), (NaCl: Wako, Cat. No. 191-01665, Na₂HPO₄: Wako, Cat. No. 197-02865, Lot ASF2635, KCl Wako, Cat. No. 163-0334T, Lot CEQ7122, KH₂PO₄: Wako, Cat. No. 169-0425, Lot ELG7616)) and centrifuged twice for 3 minutes at 3000 rpm in order to remove supernatant. Cells after washing were suspended in a solution of 1% BSA (Wako Cat No. 013-07492 Lot PKH3483)—PBS and adjusted to a cell density of 5×10⁶ cells/ml.

A primary antibody, which is a test antibody or a positive control antibody (2B8), was respectively injected in 15, 30, 50, 75, 100, 125, 150, 200 ng (1.5 to 5 μl) lots into a 1.5 ml tube (BM Equipment Co., Ltd., BM-ring lock tube, Cat. No. BM-15). At the same time, four tubes not containing antibody were prepared. Three samples were prepared for each test antibody, mixed well with 100 μl (5×10⁵ cells) of a suspension of 1% BSA (Wako Cat No. 013-07492 Lot PKH3483)—PBS, and shaken and reacted at room temperature for one hour.

After reacting, centrifugal separation was performed at 3,000 rpm for 3 minutes at room temperature using a low-temperature high-speed refrigerated centrifuge LX-100 (TOMY). After recovering the cells, the cells were suspended in 200 μl of PBS to remove unreacted primary antibody remaining on the cell surface and then centrifuged at 3,000 rpm for three minutes to remove the supernatant. This operation was repeated twice.

FITC-labeled anti murine IgG (H&L) secondary antibody [GOAT Anti-murine IgG (H&L) Fluorescein conjugated, affinity purified Secondary antibody, Chemicon, Cat. No. AP124F, Lot 24021014] was added in excess (500 ng) with respect to cell-conjugated primary antibody together with 100 μl of 1% BSA-PBS (500 ng/100 μl). The mixture was shaken for one hour at room temperature while shielding against light and primary antibodies binding to cells were detected. After the reaction, the mixture was centrifuged at 3,000 rpm for three minutes and the cells were recovered. The cells were suspended in 200 μl of PBS to remove unreacted FITC-labeled anti murine IgG (H&L) antibody remaining on the cell surface and then centrifuged at 3,000 rpm for three minutes to remove the supernatant. This operation was repeated twice.

The recovered cells were suspended in 100 μl of PBS and transferred to a flat-bottomed 96 well plate (Sumitomo Bakelite Co., Ltd., ELISA PLATE Cat. No. 8496F). The fluorescent intensity of the secondary antibodies was measured using a Typhoon9210 image analyzer (Amersham Bioscience) under the following conditions: Fluorescence mode: 600 V, 526SP/green (532 nm) Focus: bottom face+3 μm. At the same time, the controls for the preparation of a standard curve were prepared using 100 μl of PBS supplemented with 0, 12.5, 25, 50, 75, 100, 125, 150 ng of FITC-labeled secondary antibody.

After the detection step, the image was digitized using image analysis software Image Quant (Amersham Bioscience) and analyzed using Excel (Microsoft). Background values for the plate, the PBS solution and FITC-labeled secondary antibody displaying non-specific binding to cells were determined. Then a value for the reaction only between the cells and the FITC-labeled secondary antibodies was obtained. Average values for those four points were subtracted from the fluorescent intensity value for each sample in order to obtain the amount of fluorescence of cell-conjugated FITC-labeled secondary antibody. A standard curve was prepared by measuring the amount of fluorescence at various concentrations of control cell-conjugated FITC-labeled secondary antibodies. Thus the amount of cell-conjugated secondary antibody (number of moles or weight) was calculated. The amount of cell-conjugated primary antibody was calculated assuming that each primary antibody and the FITC-labeled secondary antibody react at a ratio of 1:2. Primary antibody in suspension was calculated by subtracting the cell-conjugated amount from the added amount. When calculating the antibody concentration as a molar concentration, the molecular weight of the monoclonal was taken to be 150,000.

Saturation of the binding reaction resulting from increasing addition of primary antibody was confirmed when the fluorescent intensity reached a constant value. Scatchard analysis was used to calculate the antigen number and dissociation constant (Kd value) refer to Scatchard, G.; Ann. N.Y. Acad. Sci., 51: 660-672, 1949, New Cultured Cell Experimental Methods in Molecular Biology Research, Yodosha Co., Ltd., Jikken Igaku separate volume, BioManual UP Series Revised 2^{nd} Edition, pages 212 to 217). The values were an average of three values for each sample.

The measurement results for an example of a representative monoclonal antibody producing 8 clones and a positive control antibody (2B8) are shown below in Table 3.

### (2) Biological Characteristics Tests

(a) Apoptosis Induction Test

The apoptosis induction capacity of a test antibody was measured using flow cytometry (Annexin V/PI staining). A positive control (238) and a negative control (Anti-CD3 monoclonal antibody (BD PharMingen) were used. The test was performed using a MEBCYTO Apoptosis Kit (MBL, Cat. No. 4700, Lot. 20).

After centrifuging, Raji cells were suspended in fresh RPMI1640 medium (Sigma, Cat. No. R8758, Lot. 44K2416) supplemented with 10% FBS (immobilization agent) (ICN, Cat. No. 2916754, Lot. 8005C). 1 ml of the solution with a concentration 5×10⁵ cells/ml was introduced to each well of a 12-wellplate. 12 wells were used for each antibody and each antibody was added to make a final concentration of 2 μg/ml or 4 μg/ml (3 wells×two concentrations×2 times, total 12 wells). On the first and second day after commencement of culturing, culture medium containing about 2×10⁵ cells was recovered and after centrifuging, the cells were washed once in PBS. Then the cells were suspended in 85 μl of binding buffer. After mixing well with 10 μl of Annexin V-FITC and 5 μl of PT, the mixture was allowed to react for 15 minutes at room temperature while shielding from the light. Flow cytometry measurements were performed (FACS Calibur, Becton Dickinson) and analyzed CellQuest (Becton Dickinson).

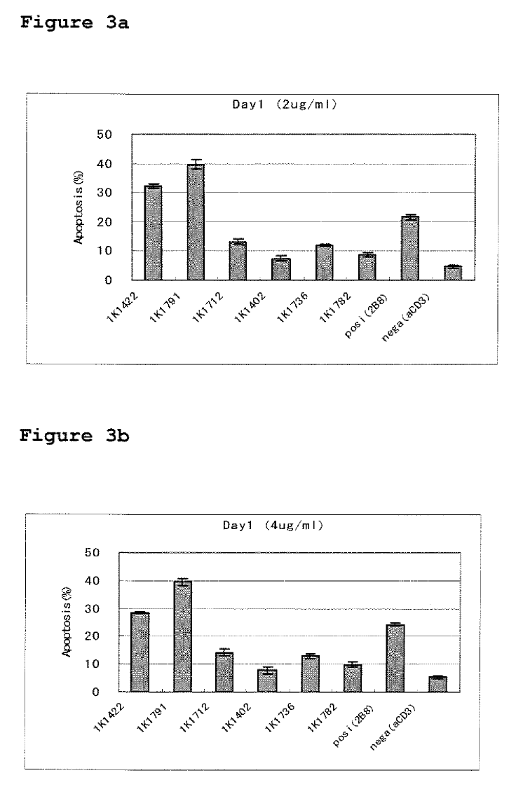

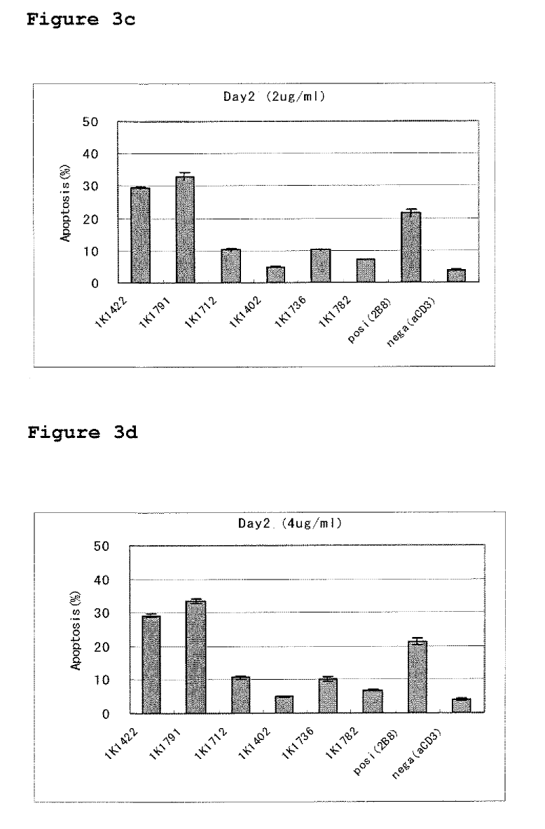

The measurement results for an example of a representative monoclonal antibody producing 6 clones, a positive control antibody (2B8) and a negative control antibody (Anti-CD3) are shown below in FIG. 3a to FIG. 3d. Although generally 2B8 was expected to have high apoptosis inducing capacity, the monoclonal antibody producing the clone 1k1791 obtained by cell fusion of the 1K17 series (immunized with CD20/CHO and Raji cells) and the clone 1k1422 obtained by cell fusion of the 1K14 series (immunized with SB cells and CD20/CHO cells) displayed a high apoptosis inducing capacity when compared with 2B8.

### (b) Cell Growth Inhibiting Tests

A Raji cell suspension having a cell concentration of 5×10⁴ cells/ml was supplemented with RPM11640 supplemented with 10% FCS. The resulting solution was added in 100 μl/well lots to a 96-wellplate and cultured. After 24 hours, culturing was continued after adding 50 μl/well of the respective antibody solutions to each well so that the antibody concentration was 1 μl/ml. 72 hours after adding the antibody, 10 μl/well of color development Cell Counting Kit-8 (Donin Kagaku, Cat. No. 343-07623, Lot. SG076) was added, culturing was continued for another 4 hours and then absorption was measured at 492 nm.

The absorbance measurement results for a monoclonal antibody producing 6 clones, a positive control antibody (2B8) and a negative control are shown below in Table 2 and their characteristics are shown in Table 3.

### Cell Growth Inhibiting Tests

TABLE 2

| Clone name | Absorption (492 nm) |
|---|---|
| 1K1422 | 1.775 |
| 1K1791 | 1.794 |
| 1K1712 | 2.326 |
| 1K1402 | 2.540 |
| 1K1736 | 2.239 |
| 1K1782 | 2.603 |
| Positive | 1.759 |
| control (2B8) | |
| Negative | 2.607 |
| control | |

### Characteristics of Monoclonal Antibodies

TABLE 3

| | | | | Cell Growth |
|---|---|---|---|---|
| | | Binding | Capacity | Inhibiting |
| | | affinity Kd | to Induce | Action (in |
| Clone name | Isotype | value (nM) | Apoptosis | Vitro) |
| 1K1422 | IgG1, κ | 3.39 | 130 | present |
| 1K1791 | IgG1, κ | 1.70 | 160 | present |
| 1K0924 | IgG2b, κ | 1.35 | 60 | present |
| 1K1712 | IgG2a, κ | 0.84 | 50 | — |
| 1K1402 | IgG1, κ | 0.78 | 30 | — |
| 1K1736 | IgG2b, κ | 0.54 | 50 | — |
| 1K1782 | IgG1, κ | 0.40 | 30 | — |
| 1K1228 | IgG1, κ | 0.26 | 30 | — |
| Positive | IgG1, κ | 6.79 | 100 | present |
| control (2B8) | | | | |

### (c) Antibody-Dependent Cell-Mediated Cytotoxicity (ADCC)

In this experiment, effector cells were activated using the Fc region of an anti CD20 chimerized antibody in order to measure the capability to lyse lymphoma cell lines.

Four types of cells, Raji, WiL2-NS, SU-DHL4 and RC-K8 derived from human B cells were cultured and incubated in RPMI1640 supplemented with inactivated 10% FCS (culturing medium) at 370C under an atmosphere of 5% CO₂.

During the experiment, the cells were washed in RPMI1640 supplemented with 10% FCS. Untreated cells took up calcein for 15 minutes during the reaction at 37° C. and then the number of cells was adjusted to 4×10⁵ cells/ml. Since calcein is only found on cells having a normal cellular membrane, it is possible to stain only normal cells. Rituximab (C2B8) which is an anti 0020 chimerized antibody and the 6 types of chimerized antibody (1k0924, 1k1402, 1k1422, 1k11712, 1k1736, 1k1791) were adjusted to 20 μg/ml, 4 μg/ml and 0.8 μg/ml using RPMI1640 supplemented with 10% FCS. Blood was taken from a healthy subject immediately layered onto a Ficoll and centrifuged in order to extract a lymphocyte fraction which was then adjusted to 5×10⁶ cells/ml, 1×10⁶ cells/ml and 0.2×10⁶ cells/ml.

25 μl of cells with an adjusted concentration, 25 μl of each anti CD20 chimerized antibody solution (having respective final concentrations of 5 μg/ml, 1 μg/ml, 0.2 μg/ml) and 50 μl of effector cells at each antibody concentration (having respective E:T ratios of 25:1, 5:1 and 1:1) making a total of 100 μl was mixed well in a 96-wellplate and reacted for 4 hours in an incubator at 37° C. under an atmosphere of 5% CO₂. In order to calculate the natural lysis of cells, three samples were prepared: a sample in which the antibody solution and the effector cells are replaced by RPMI1640 supplemented by 10% FCS, a sample in which the antibody solution is replaced by RPMI1640 supplemented by 10% FCS which is a sample for calculating the activity of only the effector cells independently of the presence of antibodies, and a sample in which the antibody solution is replaced by 20% TritonX-100 which is a sample for calculating the maximum lysis.

After the reaction, since calcein is discharged out of the cells as a result of the rupture of the cellular membrane when the cell lyses, Quencher was used to quench the fluorescence of calcein suspended in the reaction solution and then fluorescence was measured using a fluorescence analyzer.

After analysis, the image was digitized using image analysis software (Amersham Bioscience) in order to calculate a lysis rate For each sample using the equation below.

Lysis rate %=((Natural lysis)−(Sample))/((natural lysis)−(Maximum lysis))×100 Equation 1

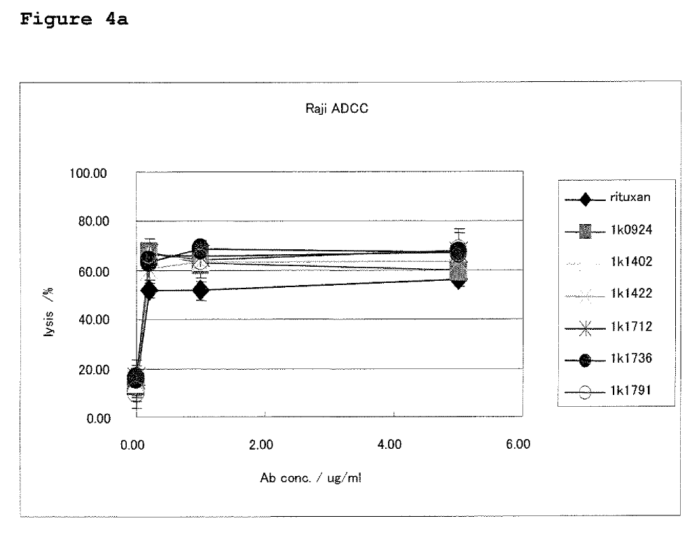

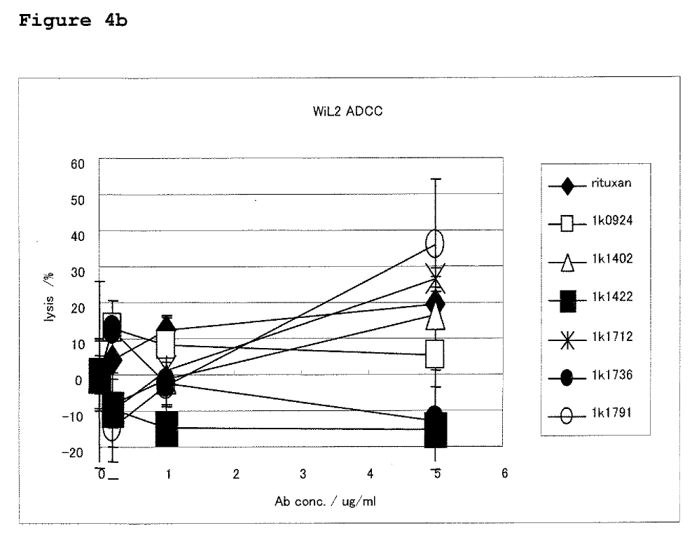

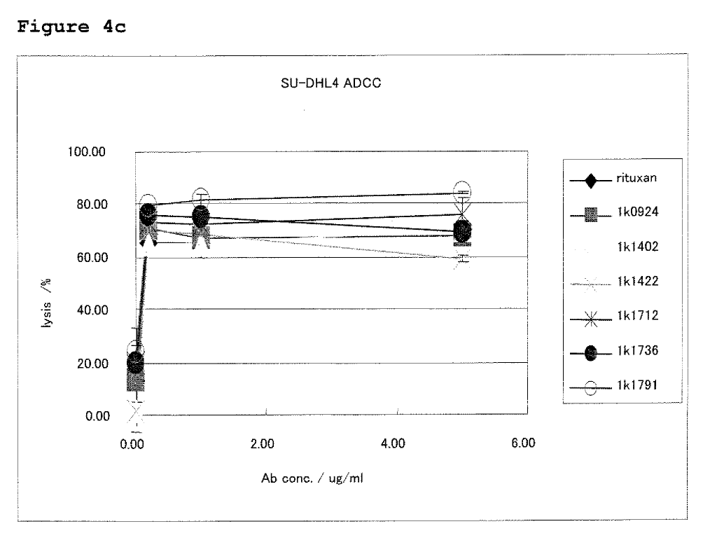

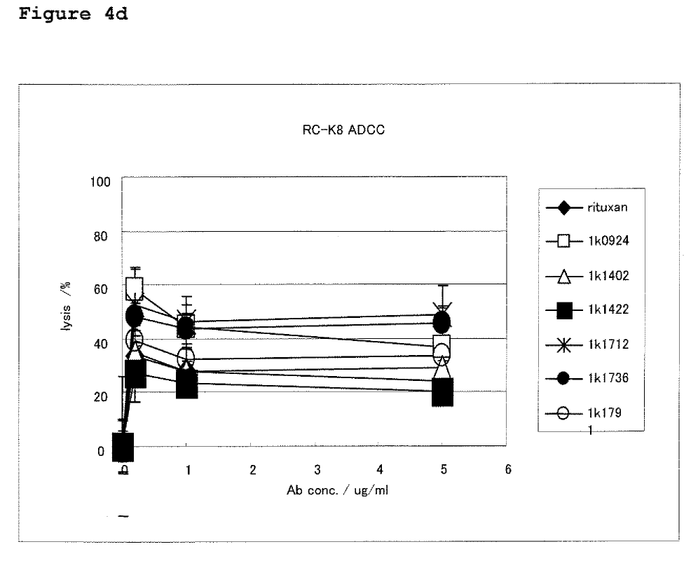

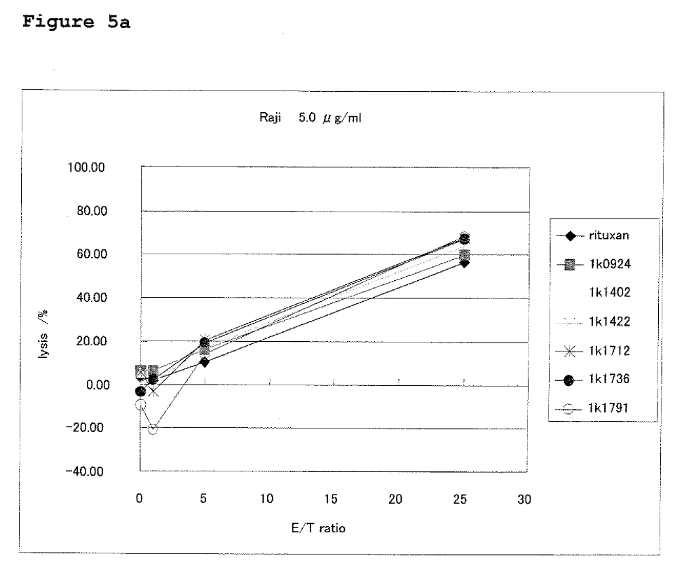

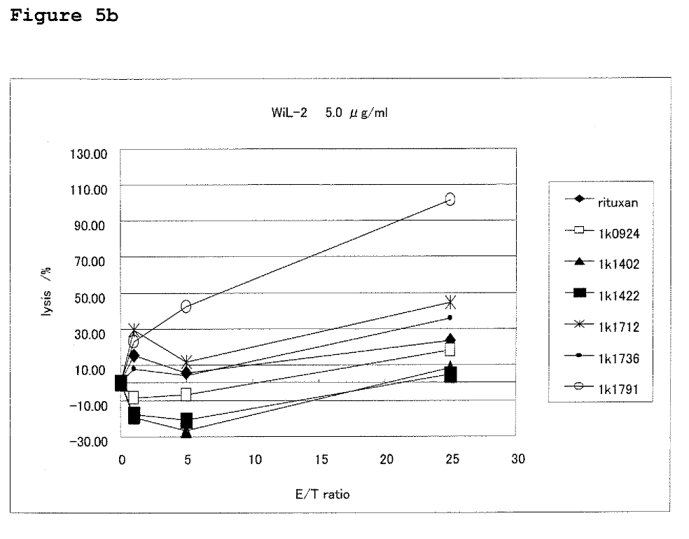

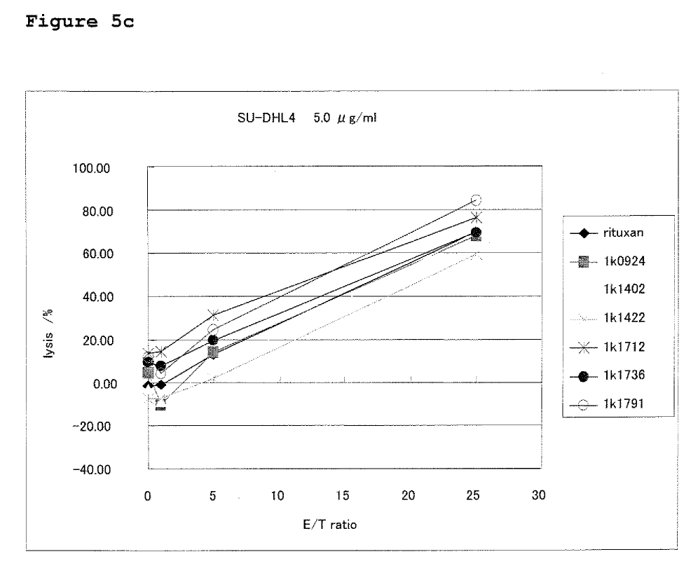

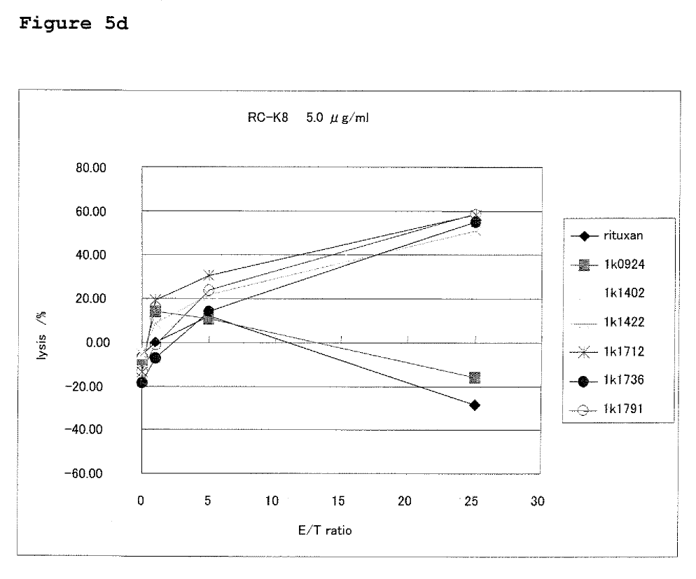

The relationship between antibody concentration and cytotoxicity for each cell type when the ratio (E:T ratio) of the number of target cells to the number of effector cells such as NK cells is 25:1 is shown in FIG. 4a to FIG. 4d. The relationship between cytotoxicity and the E:T ratio for each cell type when the antibody concentration is 5 μg/ml is shown in FIG. 5a to FIG. 5d.

As shown in FIG. 4a to FIG. 4d, when the E:T ratio is 25:1, each cell shows cytotoxicity when antibodies are added. In other words, the antibodies participate in cytotoxicity. Furthermore cell strains other than WiL2-NS display activity equivalent to 1 μg/ml and 5 μg/ml at an antibody concentration of 0.2 μg/ml (saturation occurs at 0.2 μg/ml). Activity becomes constant after reaching a maximum and effects are evident at an antibody amount which is less than the antibody required for complement dependent cytotoxicity.

As shown in FIG. 5a to FIG. 5d, the effect of the E:T ratio on cytotoxicity when the antibody concentration is 5 μg/ml is that cytotoxicity increases in an E:T ratio-dependent manner. This shows that cytotoxicity occurs as a result of effector cell action.

### (d) Complement Dependent Cytotoxicity (CDC)

In this experiment, measurements were made of the capability of anti CD20 chimerized antibodies to lyse lymphoma cell lines in the presence of serum containing complement.

Four types of cells, Raji, WiL2-NS, SU-DHL4 and RC-K8 derived from human B cells were cultured and incubated in RPMI1640 supplemented with inactivated 10% FCS (culturing medium) at 37° C. under an atmosphere of 5% CO₂.

During the experiment, the cells were washed in RPMI1640 supplemented with 10% FCS and the cell number was adjusted to 2−3×10⁶ cells/ml. C2B8 (rituximab) which is an anti CD20 chimerized antibody and the 6 types of chimerized antibody (1k0924, 1k1402, 1k1422, 1k1712, 1k1736, 1k1791) were adjusted to 20 μg/ml, 4 μg/ml and 0.8 μg/ml using RPMI1640 supplemented with 10% FCS.

55 μl of cells with an adjusted concentration, 25 μl of each anti CD20 chimerized antibody solution (having respective final concentrations of 5 μg/ml, 1 μg/ml, 0.2 μg/ml) and 20 μl of pooled serum taken from five healthy subject or an inactivated type thereof making a total of 100 μl was mixed well using a vortex mixer and reacted for 2 hours in an incubator at 37° C. under an atmosphere of 5% CO₂. A sample in which 25 μl of antibody solution is replaced by RPMI1640 supplemented with 10% FCS was prepared as a sample for background calculations.

After the reaction, PI (propidium iodide) was used to stain dead cells and analysis was performed using a FACS (Becton Dickinson) The numerical results used the population of dead cells without modification and subtracted the background values and the sample supplemented with inactivated serum.

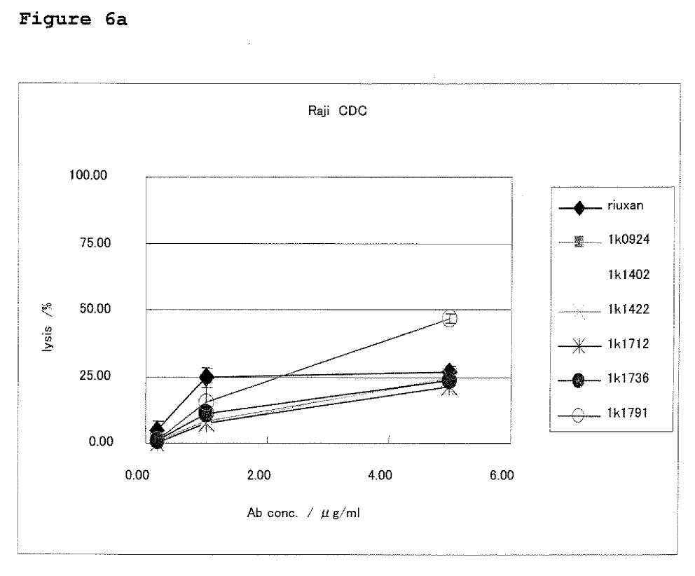

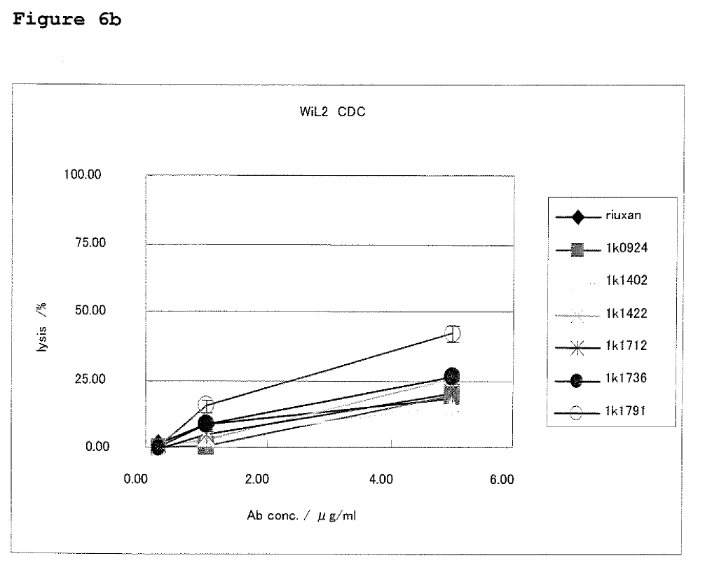

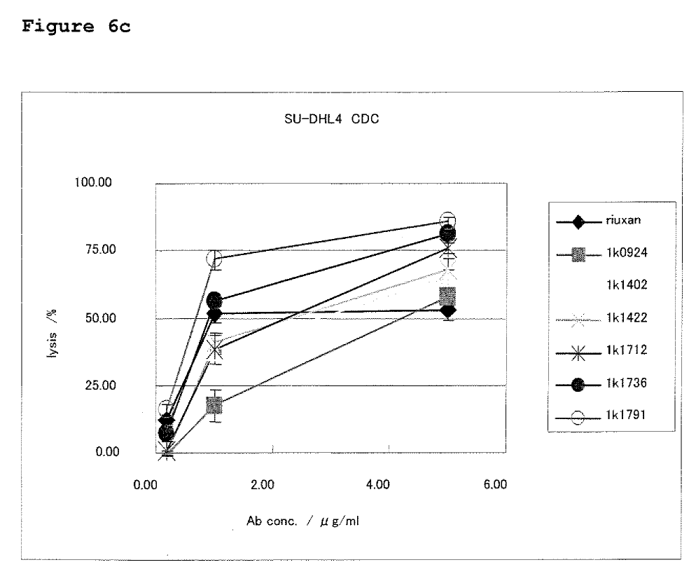

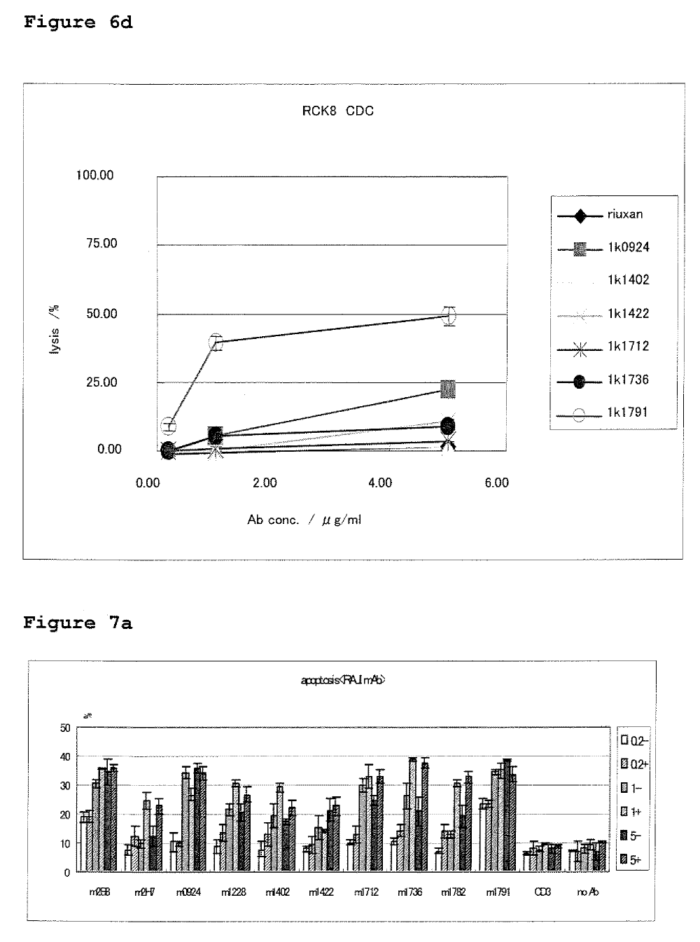

The relationship between antibody concentration and cytotoxicity for each cell type is shown in FIG. 6a to FIG. 6d.

As shown in FIG. 6a to FIG. 6d, all six types of antibodies show activity in Raji, WiL2-NS and SU-DHL4. Furthermore, although concentration dependency can be confirmed, when concentrations at 5 μg/ml are compared, 1k1791 shows a particularly high activity in comparison to the other antibodies. In addition, 1k1736, 1k1422 and 1k1712 show high activity. At this concentration, 1k1791 induces approximately twice the cytotoxicity with respect to WiL2-NS cells in comparison to other antibodies. However the other antibodies also display equal or greater activity than rituximab.

RC-K8 cells on which rituximab has no effect shows very clearly the difference between the respective antibodies. Rituximab, 1k1402 and 1k1712 show no activity or almost no activity. In contrast, 1k1791 shows extremely high activity, and at a concentration of 5 μg/ml shows cytotoxicity of approximately 50%. Thereinafter 1k0924 shows cytotoxicity of approximately 25% and 1k1422 and 1k1736 show cytotoxicity of approximately 10%.

From the above, it can be confirmed that the 6 types of chimerized antibodies which are the subject of the present test display CDC activity which is equal to or stronger than rituximab.

### Example 3

### (1) Measurement of Binding Affinity

Raji cells derived from human B cells were cultured in a CO₂ incubator at 37° C. in an atmosphere of 5% CO₂ using RPMI1640 medium supplemented with inactivated 10% FCS. The cells were passaged twice per week.

Culture solution on three to four days after subculturing containing (approximately 1×10⁶ cells/ml) cells was centrifuged for five minutes at 1,000 rpm at room temperature. The cells were recovered, suspended in PBS (−) and centrifuged for five minutes at 1,000 rpm in order to remove supernatant. This operation was performed twice and then the cells were washed.

The primary antibody reaction was performed by mixing well an anti CD20 antibody (positive control antibody: antibody 2B8, chimerized antibody and humanized antibody C2B8) with Raji cells and reacting the mixture at room temperature for one hour. The respective final concentrations of anti CD20 antibodies has twelve values of 1.33, 2.67, 4.00, 5.33, 6.67, 8.00, 9.33, 10.67, 12.00, 13.33, 14.67, 16.00 nM. The reaction solution was 1% BSA-PBS solution with a cell number of 5×10⁶ cells and was injected into a 1.5 ml tube to a final volume of 100 μl. Three samples for each test antibody were prepared and four tubes to which antibody was not added were prepared as samples for background calculations.

After reacting, the mixture was centrifuged at room temperature for three minutes at 3,000 rpm in order to remove unreacted primary antibody and the cells were recovered.

FITC-labeled secondary antibody was adjusted to have a concentration of 5 μg/ml in 1% BSA-PBS solution. This solution was added in 100 μl lots so as to be in excess with respect to the primary antibody binding to the cells. After suspending and shielding against the light, the solution was reacted for one hour at room temperature.

When the FITC-labeled secondary antibody used was a murine antibody, the antibody was GOAT Anti-murine IgG (H&L)-FITC. When the secondary antibody was a chimerized or humanized antibody, the antibody was GOAT F(ab′) 2 Fragment Anti Human IgG (Fcγ)-FITC.

After the reaction, the mixture was centrifuged at 3,000 rpm for three minutes at room temperature. Unreacted FITC-labeled secondary antibody was removed and the cells were recovered. The cells were suspended in 200 μl of PBS centrifuged again and washed.

The cells were suspended in 100 μl of PBS and transferred to a flat-bottomed 96-well plate. The fluorescent intensity of the secondary antibodies was measured using a Typhoon9210 analyzer (Amersham Bioscience).

After the detecting step, an image was digitized using image analysis software Image Quant (Amersham Bioscience) and analyzed using Excel (Microsoft). Average values for the same test antibodies were calculated and the values for background calculation samples (when only reacting a cell with a FITC-labeled secondary antibody) were subtracted from the test antibody values. The background values derived for the FITC-labeled secondary antibody binding non-specifically to cells, the PBS solution and the plate are omitted. At the same time, a standard curve was prepared by measuring the amount of fluorescence of only FITC-labeled secondary antibodies at amounts of 0, 12.5, 25, 50, 75, 100, 125, and 150 ng per 100 μl. In this manner, the number of moles of secondary antibody binding to cells was calculated. Assuming that each primary antibody and the FITC-labeled secondary antibody react at a ratio of 1:5, the amount of cell-bound primary antibody was calculated. Primary antibody in suspension was calculated by subtracting the bound amount from the added amount. Scatchard analysis was performed using these values in order to calculate a dissociation constant Kd.

The results are shown below in Table 4.

### (2) Apoptosis Inducing Test

Initial apoptosis was detected using a Annexin V-FITC apoptosis kit using two conditions: a condition (no cross linking) in which apoptosis induced independently by anti CD20 antibodies against cells derived from human B cells stains, and a condition (cross linking) in which apoptosis was induced by adding secondary antibodies recognizing the Fc region of the anti CD20 antibody.

Four types of cells, Raji, WiL2-NS, SU-DHL4 and RC-K8 derived from human B cells were cultured by incubating in RPMI1640 supplemented with inactivated 10% FCS (culturing medium) at 370C under an atmosphere of 5% CO₂. The cells were subcultured twice per week.

Three to four days after subculturing, culture solution (approximately 1×10⁶ cells/ml) was centrifuged for five minutes at 1,000 rpm at room temperature and the cells were recovered.

Anti CD20 antibodies (positive control antibody: murine antibody 2B8, chimerized antibody and humanized antibody C2BB, negative control: Anti-CD2 monoclonal antibody) were mixed well with cells suspended in fresh culture medium and reacted in an incubator at 37° C. under an atmosphere of 5% CO₂. The final concentrations of the anti CO₂₀ antibodies were 0.2, 1 and 5 μg/ml. The culture medium with a cell concentration of 1×10⁶ cells was used as the reaction solution and was reacted in a 1.5 ml tube to a final volume of 250 μl. Three samples were prepared for each test antibody.

After the reaction, the mixture was centrifuged at 1,200 rpm for three minutes at room temperature, unreacted antibody was removed and the cells were recovered.

Under the no cross linking condition, fresh culturing media was used, under the cross linking condition, five times the amount of the CD20 antibody of a secondary antibody recognizing the Fc region was added in 250 μl lots. After mixing well, the mixture was reacted for three more hours in an incubator at 37° C. in an atmosphere of 5% CO₂. When secondary antibody used was a murine antibody, the antibody was GOAT Anti-murine IgG Fcγ Fragment and when the secondary antibody was a chimerized or humanized antibody, the antibody was GOAT Anti Human IgG Fcγ-Fragment specific.

After the reaction, the mixture was centrifuged at 3,000 rpm for three minutes at room temperature. Unreacted secondary antibody was removed and the cells were recovered. The cells were suspended in 250 μl of PBS, centrifuged again and washed.

Test reagents from a MEBCYTO apoptosis kit-AnnexinV-FITC, PI-(MBL, Cat. No. 4700, Lot. 21) were used. After suspending the cells in 85 μl of Binding buffer, 5 μl of Annexin V-FITC and 5 μl of propidium iodide (PI) (to a final concentration of 0.5 mg/ml) were added and mixed well. The mixture was shielded from the light and allowed to react at 15 minutes at room temperature.

A total count 20,000 of cells was measured using flow cytometry (EPICS ALTRA: BECKMAN COULTER) and analyzed (Expo32: BECKMAN COULTER).

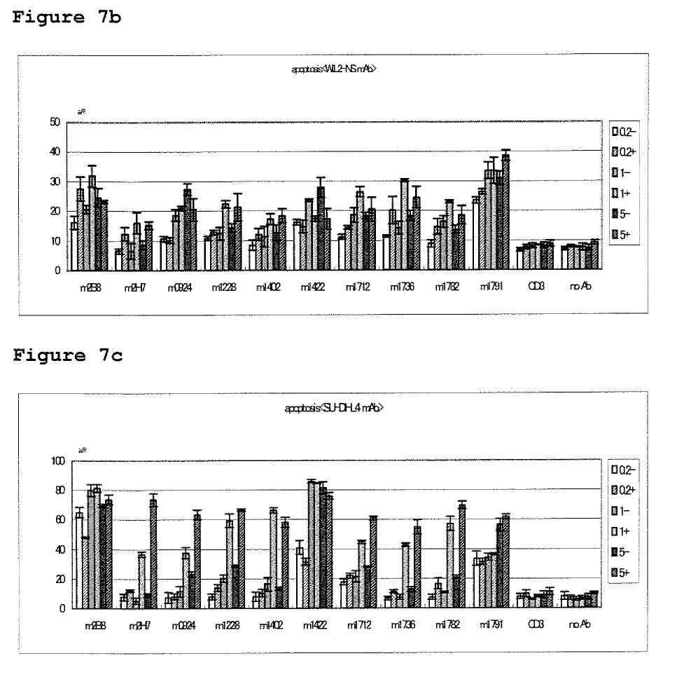

The results are shown in FIG. 7a to FIG. 9d.

### (3) Preparation of Humanized Antibody Producing Strain

### (a) DNA Synthesis

DNA optimized to CHO cells with a codon based on amino acid sequences in SEQ ID Nos: 17 to 24 were designed and synthesized.

### (b) Preparation of the Construct

16 types of humanized 1K1791 expression constructs were prepared using pNOW as an expression vector. pNOW-aa1791kg1, pNOW-af1791kg1, pNOW-as1791kg1, pNOW-av1791kg1, pNOW-fa1791kg1, pNOW-ff1791kg1, pNOD-fs1791kg1, pNOW-fv1791kg1, pNOW-sa1791kg1, pNOW-sf1791kg1, pNOW-ss1791kg1, pNOW-sv1791kg1, pNOW-va1791kg1, pNOW-vf1791kg1, pNOW-vs1791kg1, pNOW-vv1791kg1

### (c) Transfection and Selection Using Chemical Reagent

A humanized 1K1791 expression construct was introduced Into CHO DG44cdB cells using a transfection reagent. 1×10⁶ CHO DG44cdB cells containing the respective genes were suspended in 100 μl of selective medium, dispersed on five 96-wellplates (200 μl/well) and cultured for 3 to 4 weeks at 37° C. under an atmosphere of carbon dioxide gas.

Transfection reagent: Qiagen, Effectene Transfection Reagent, Cat. No 301427.

Selective Medium: IS CHO-CD w/Hydrolysate/4 mM GlutaMAX/0.8 mg/ml G418.

### (d) Selection of High Expression Cell Strain

1) Supernatant was recovered from wells where colonies were present and the antibody production amount was measured using a Dot Blot assay.
2) Clones displaying a high antibody production amount were transferred to a 24 wellplate and after culturing for approximately 5 days, the supernatant was recovered and the antibody production amount was measured using a Sandwich ELISA
3) Two clones displaying a high antibody production amount were selected and transferred to a T75 flask.

### (e) Small Scale Culturing

The two selected clones were cultured in a T75 flask containing 30 μl of selective medium against the 16 types of constructs.

### (4) Culturing and Purification of Humanized Antibody Producing Strains

Antibody producing cell strains (genetically recombinant CHO-DG44 cells) were cultured in IS CHO-CD/with Hydrolysate medium (Irvine Scientific, Cat. No. 91119) containing Hydrolysate supplemented with 4 nM GlutaMax (Invitrogen, Cat 35050-061) and 200 μg/ml of G418 (Sigma, Cat. No. A1720-53) in a CO₂ incubator under an atmosphere of 5% CO₂ at 37° C. The cells were passaged twice per week.

Cell culture solution approximately two weeks after subculturing was centrifuged at 3,500 rpm for five minutes at room temperature. The supernatant was recovered, filtered using a 0.45 μm syringe filter and equilibrated using 50 nM Tris-HCl, pH 7.0.

After adding supernatant to a Hi Trap Protein A HP column (GE Healthcare, Cat No. 17-0402-01), washing was performed using 50 nM Tris-HCl, pH 7.0. Elutions were obtained using 0.1M citric acid pH 4.0. 400 μl was collected on each occasion and neutralized with 40 μl (or a 10/1 amount) of 1 M Tris-HCl, pH 9.0. After dialyzing twice against a 100 times amount of PBS for 2.5 hours using a M. W. 3500 diafiltration cup (Bio-Tech Cat. No. 212932), dialysis was performed for 15 to 18 hours once.

The antibody producing strains used were CHO cells hz1791-fv10, hz1791-ff34, hz1791-sf43 and hz1791-ss32. These strains were deposited respectively under Accession Nos. FERM BP-10543, FERM BP-10544 FERM BP-10545 FERM BP-10546 with the International Patent Organism Depositary (IPOD), National Institute of Advanced Industrial Science and Technology (AIST), Tsukuba Central 6, 1-1-1 Higashi, Tsukuba, Ibaraki, Japan on 1 Mar. 2006 under the provisions of the Budapest Treaty.

The amino acid sequences (SEQ ID Nos: 17 to 24) for the H chains of the variable region and the L chain of the variable region of a humanized anti CD20 monoclonal antibody obtained from the above cell strains are described hereinafter (the underlined sections differ from the corresponding murine antibody).

Sequence for Humanized 1k1791

Amino acid sequence of L chain variable region
(SEQ ID No: 20)
Ven 1791: STVMTQSPDSLAVSLGERVTINC KASQSVSNDVA
WYQQKPGQSPKVLIY FASNRYT GVPDRFSGSGYGTDFTFTISSVQAED
VAVYFCQQDYSSPLT FGAGTKLELK
Amino acid sequence of H chain variable region
(SEQ ID No: 24)
Ven 1791: QIQLVQSGPELKKPGASVKISCNASGYTFT NFGVN
WVKQAPGKGLKWMG WINTYTGEPSYADDFKF RFAFSLDASVSTAY
LQISSLKAEDTSTYFCTR RTNYYGTSYYYAMDY WGQGTTVTVSS
Amino acid sequence of L chain variable region
(SEQ ID No: 17)
abb 1791: STVMTQSPDSLAVSLGERATINC KSSQSVSNDVA
WYQQKPGQSPKVLIY FASNRYS GVPDRFSGSGYSTDFTLTISSLQAED
VAVYFCQQDYSSPLT FGASTKLEIK
Amino acid sequence of H chain variable region
(SEQ ID No: 21)
abb 1791: QIQLVQSGSELKKPGASVKVSCKASGYTFT NFGVN
WVRQAPGKGLEWMG WINTYTGEPSYAQGFTG RFVFSLDASVSTAY
LQISSLKAEDTATYFCTR RTNYYGTSYYYAMDY WGQGTTVTVSS
Amino acid sequence of L chain variable region
(SEQ ID No: 19)
sdr 1791: STVMTQSPDSLAVSLGERATINC KSSQSNSNDVA
WYQQKPGQSPKVLIY FASNRYS GVPDRFSCSGYGTDFTLTISSLQAED
VAVYFCQQDYSSPLT FGAGTKLEIK
Amino acid sequence of H chain variable region
(SEQ ID No: 23)
sdr 1791: QIQLVQSGSELKKPGASVKVSCKASGYTFT NFGVN
WVRQAPGKGLKWMG WINTYTGEPSYAQGFTG RFAFSLDASVSTAY
LQISSLKAEDTATYFCTR RTNYYSTSYYYAMDY WGQGTTVTVSS
Amino acid sequence of L chain variable region
(SEQ ID No: 18)
fra 1791: STVMTQSPSFLSASVGDRVTITC KASQSVSNDVA
WYQQKPCQSPKVLIY FASNRYT GVPDRFSGSGYGTDFTLTISSLQAED
VAVYFCQQDYSSPLT FGAGTKLEIK
Amino acid sequence of H chain variable region
(SEQ ID No: 22)
fra 1791: QIQLVQSGSELKKPGASVKVSCKASGYTFT NFFVN
WVKQAPGKGLKWMG WINTYTGEPSYADDFKG RFAFSLDASASTAY
LQISSLKAEDMATYFCTR RTNYYGTSYYYAMDY WGQGTTVTVSS

In this experiment, average values were obtained by performing experiments with respect to each two clones from the total combination of 16 types of sequences resulting from combining the four types AbbL, FraL, SdrL, VenL x the four types AbbH, FraH, SdrH, VenH. From the above results, it is possible to create a classification of 4 groups as shown in Table 4 based on Kd values. One type was selected from each group and used in the experiment described hereinafter.

Control: c2B8
Group I: Kd=20 nM<

### Group II: Kd=10 to 20 nM

### Group III: Kd=8 to 10 nM

### Group IV: Kd=<8 nM

### Humanized Antibody Binding Dissociation Constant

TABLE 4

| Hz1791 clone No. | Kd (nM) | C2B8 Kd (nM) |
|---|---|---|
| Aa008 | 11.68 | 6.61 |
| Aa012 | 9.96 | 4.68 |
| Fa007 | 11.34 | 5.63 |
| Fa008 | 8.83 | 4.91 |
| Sa023 | 14.66 | 6.13 |
| Va016 | 13.09 | 6.67 |
| Va024 | 7.50 | 6.47 |
| Af021 | 6.84 | 5.56 |
| Af025 | 8.67 | 4.29 |
| Ff019 | 8.50 | 5.70 |
| Ff034 | 7.81 | 4.00 |
| Sf043 | 11.60 | 4.34 |
| Sf056 | 13.79 | 6.01 |
| Vf029 | 7.78 | 3.32 |
| Vf031 | 7.79 | 4.54 |
| As001 | 11.89 | 5.74 |
| As002 | 11.47 | 8.7 |
| Fs007 | 6.33 | 5.63 |
| Fs024 | 11.09 | 3.97 |
| Ss020 | 24.39 | 4.10 |
| Ss032 | 21.79 | 7.25 |
| Vs006 | 8.8 | 4.23 |
| Vs011 | 11.9 | 6.09 |
| Av004 | 10.71 | 5.76 |
| Av006 | 9.17 | 4.86 |
| Fv010 | 7.17 | 4.39 |
| Fv028 | 7.25 | 4.74 |
| Sv015 | 14.31 | 6.30 |
| Sv020 | 10.85 | 5.36 |
| Vv018 | 7.14 | 5.04 |
| Vv023 | 7.01 | 4.86 |

### Humanized Antibody Binding Dissociation Constant

TABLE 5

| Group | Hz1791 clone | Kd (nM) |
|---|---|---|
| I (20 nM <) | ss | 23.09 |
| II (10 to 20 nM) | sa | 14.66 |
| | sf | 12.70 |
| | sv | 12.58 |
| | as | 11.68 |
| | aa | 10.82 |
| | vs | 10.35 |
| | va | 10.30 |
| | fa | 10.09 |
| III (8 to 10 nM) | av | 9.94 |
| | fs | 8.71 |
| | ff | 8.15 |
| IV (<8 nM) | vf | 7.78 |
| | af | 7.76 |
| | fv | 7.21 |
| | vv | 7.07 |
| Control | c2B8 | 5.35 ± 1.13 |

The experimental results for apoptosis using 8 kinds of murine antibody are shown in FIG. 7a to FIG. 7d. We succeeded in dividing the 8 types of murine antibody and the known CD20 antibodies 2B8 and 2H7 into two types with respect to the four types of cell (omitting some exceptions.

Group A: m0924, m1422, m1791, m2B8
Group B: m1228, m1402 m1712, m1782, m2H7
(However, m0924 is included in Group B with respect to SU-DHL4 cells).

In other words, anti CD20 antibodies belonging to Group A display sufficient capability to induce apoptosis independently and display approximately the same level of apoptosis inducing capability even under cross linking conditions with a secondary antibody. However Group B displays a large increase under cross linking conditions. Furthermore the affinity of antibodies belonging to Group A is equal to that of 2B8 antibody while the affinity of antibodies belonging to Group B display an affinity higher than that of 2B8. Consequently it can be inferred that Group A is more suitable for pharmaceutical use since Group A can induce apoptosis independently and does not require the presence of a secondary antibody to induce apoptosis.

Turning now to cell type, the ratio of apoptosis for RAJI, WIL2-NS, RCKS has maximum values on the level of 30 to 40%. In contrast, SU-DHL4 displays a value of more than 80%.

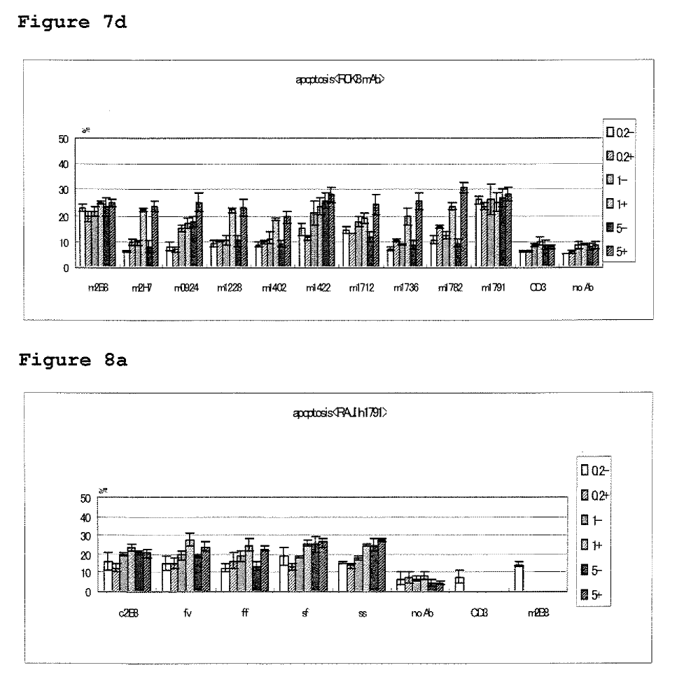

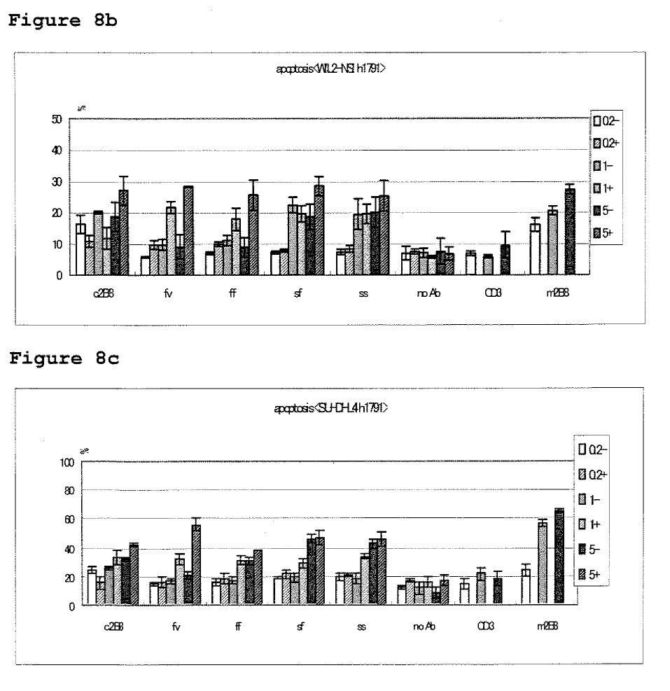

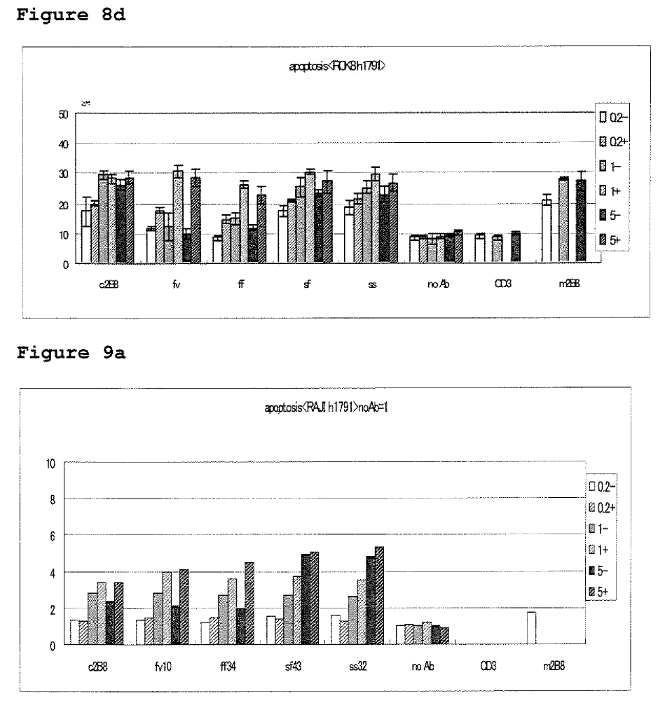

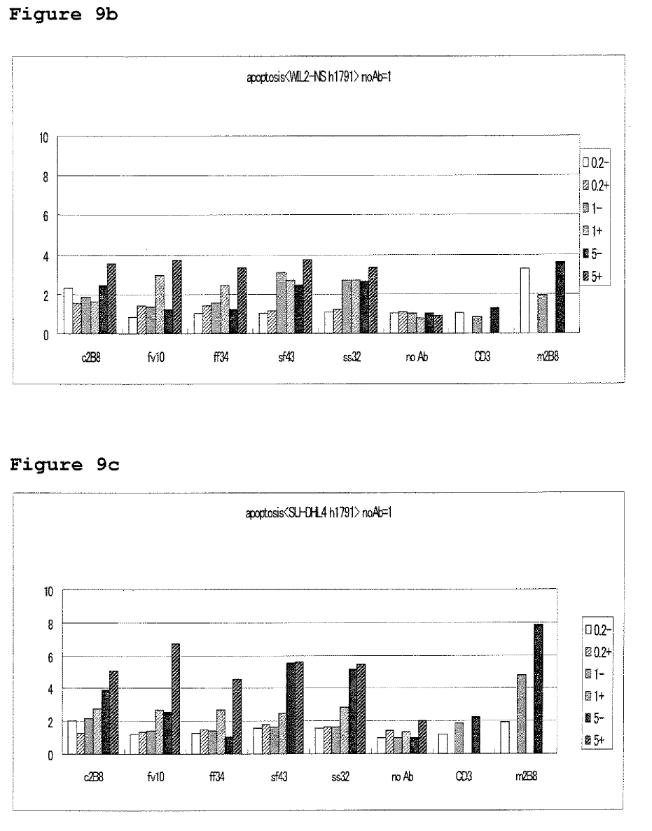

The results for the four types of humanized antibodies are shown in FIG. 8a to FIG. 9d.

In the same manner as murine antibodies, the four types of humanized 1791 antibody are classified into two types with respect to the four types of cell.

### Group A: fv, ff

### Group B: sf, ss, C2B8

The antibodies fv, ff of Group A which have an equal affinity to that of C258 show almost no apoptosis activity under no cross linking condition and have clear activity under cross linking condition. The sf, ss antibodies of Group B display a greater dissociation constant and weaker affinity than the antibodies of Group A. These antibodies independently display apoptosis activity that is stronger than C2B8.

When anti CD20 antibodies independently display a sufficient capability of inducing apoptosis against B cells, the ratio under cross linking conditions is substantially the same. However when anti CD20 antibodies do not display a sufficient capability of inducing apoptosis due to antibody type or lack of affinity conditions, it is hypothesized that apoptosis activity will increase under cross linking conditions.

FIG. 9a to FIG. 9d are graphs showing early apoptosis (%) with a value of 1 when antibody is not added on the day of the experiment.

### Example 4

Relationship of Binding Dissociation Constant (Kd value) and Cell Growth Inhibiting Properties of Humanized Antibodies

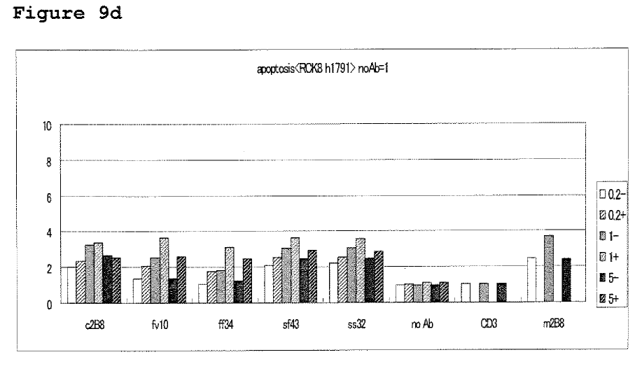

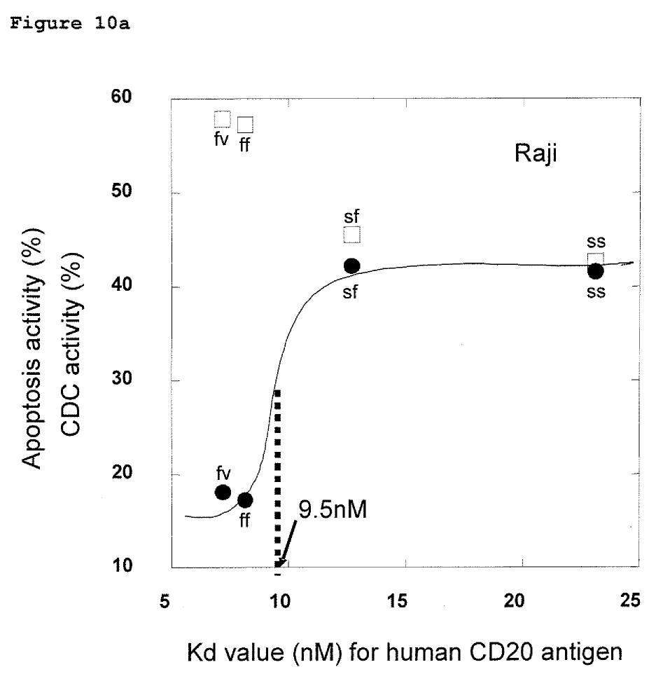

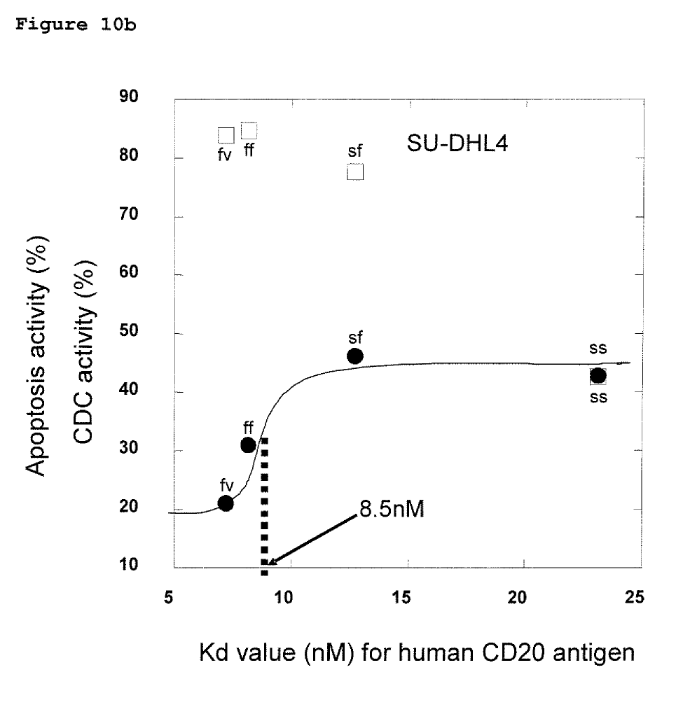

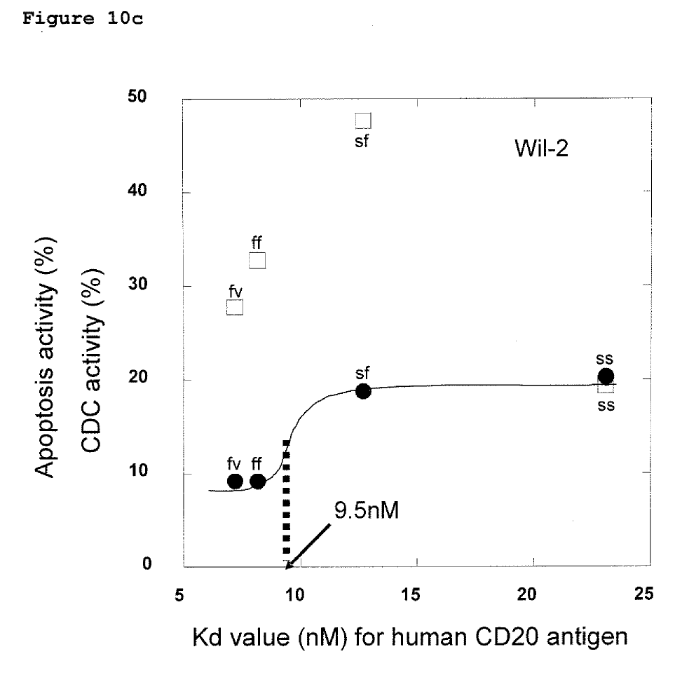

On the basis of the division of humanized antibodies into Group A and Group B in Example 3, the relationship between Kd value (nM) with respect to human CD20 antigen, apoptosis inducing activity (5) and CDC activity (%) was examined with respect to the respective hz1791 clones shown in Table 5. The measurement of the Kd value was performed in the same manner as Example 2. The measurement of CDC activity was performed in the same manner as Example 2. (However in experiments examining the relationship between CDC activity and antibody amount, the antibodies were created in the manner described hereinafter). The measurement of ADCC was performed in the same manner as Example 2. The measurement of apoptosis activity was performed in the same manner as Example 3. B cells used in the experiments were Raji, SU-DHL4, WiL-2 and RCKS. Data regarding the four clones fv, ff, sf and ss for the respective cells are shown in FIG. 10a to FIG. 10d.

The same tendency was observed with respect to apoptosis activity for all cells used in the experiment. In other words, the higher the affinity (the smaller the Kd value) of an antibody, the lower the apoptosis inducing activity. The lower the affinity (the larger the Kd value) of an antibody, the higher the apoptosis inducing activity. Furthermore when the Kd value exceeds 13 nM, apoptosis activity tends to reach a constant value. Antibodies displaying high affinity do not display apoptosis activity independently and require cross linking using a secondary antibody in order to induce apoptosis. Antibodies displaying low affinity display apoptosis activity independently.

When using Raji and SU-DHL4 cells, CDC activity was observed to increase as the affinity (Kd value is small) increased. In contrast, when using WiL-2 and RCK8 cells, the Kd value displayed a tendency to reach an extremely high value near to 13 nM.

On the basis of the observations above, the following two selection criteria were determined to allow identification of candidate antibodies for use as pharmaceuticals:

Although the antibody does not induce apoptosis independently, affinity to CD20 antigen is extremely high and the antibody may display anti-cancer activity through CDC activity;

The antibody induces apoptosis Independently, and from among the antibodies which may display anti-cancer activity through CDC activity and apoptosis, the antibody displays high affinity to CD20 antigen.

The former selection criterion is preferred when selecting antibodies which display high affinity to CD20 antigen and have CDC activity (a tendency for CDC activity to increase as the Kd value decreases). Since antibodies selected on this basis do not induce apoptosis independently, only the value for CDC activity is of concern. Clones satisfying the former selection criterion do not induce apoptosis in isolation, but have extremely high values for CDC activity and affinity and therefore promote cell deletion.

In the latter criterion, it is preferred to select antibodies in which the total of apoptosis and CDC activity of the antibody is high and which have high affinity to CD20 antigen. Thus although clones satisfying the latter criterion do not display high affinity, the antibodies have high apoptosis activity and therefore these antibodies promote cell deletion via the synergistic effect of inducing apoptosis and CDC.

In order to determine boundary points for the two selection criteria above, Kd values giving the midpoint for apoptosis activity of the four cell types were represented graphically (the dotted line in the graphs in FIG. 10a to FIG. 10d). There is not a large difference in the Kd values giving the midpoint for apoptosis activity in these four graphs. The value for Raji cells is 9.5 nM, SU-DHL4 cells is 8.5 nM, WiL-2 cells is 9.5 nM and RCK8 cells is 10 nM. These results allowed the boundary point for the above two selection criteria to be set at a value of approximately 9.5 nM.

The upper limit for the Kd value in the latter selection criteria was set at 13 nM being in a range wherein the total of apoptosis activity and CDC activity is as large as possible and moreover affinity is high (Kd value is small) taking into account the extremely high value for CDC activity seen in WiL2 cells and RCK8 cells (FIG. 10c and FIG. 10d).

Thus according to the former selection criterion, the clones ff and fv are selected as antibodies which have a Kd value for human CD20 antigen of less than approximately 9.5 nM, have a Kd value as small as possible and high CDC activity (FIG. 10a and FIG. 10b). According to the latter selection criterion, the clone sf is selected as an antibody which has a Kd value for human CD20 antigen of approximately 9.5 nM to approximately 13 nM, has a total of apoptosis activity and CDC activity as high as possible and has a small Kd value (FIG. 10c and FIG. 10d).

CDC activity and apoptosis activity were measured by selecting fv, ff, sf and ss from the four types of humanized clones and using Raji cells, SU-DHL4 cell, WiL-2 cells and RCKS cells. The results are shown in Table 6 and Table 7. Rituximab (C2B8) and the clone 1791 (c1791) were contrasted.

CDC Activity of Humanized Antibodies according to the Present Invention

TABLE 6

| | CDC (10 ug/ml) | | | |
|---|---|---|---|---|
| | RAJI | WIL2NS | SUDHL4 | RC-K8 |
| Antibody | c2B8 = 100 | c2B8 = 100 | c2B8 = 100 | c2B8 = 100 |
| c2B8 | 100 | 100 | 100 | 3 |
| fv | 99 | 172 | 120 | 498 |
| ff | 98 | 203 | 121 | 530 |
| sf | 78 | 295 | 111 | 713 |
| ss | 73 | 120 | 61 | 108 |
| c1791 | 99 | 529 | 119 | 100 |

The results in Table 6 show that the clones ff and fv display a CDC activity which is equal to or stronger than the activity of rituximab. Furthermore sf shows extremely strong CDC activity with respect to WIL2 and RCKS cells.

An experiment was conducted to investigate the relationship between CDC activity and antibody concentration. The antibodies used were more highly purified than the antibodies treated according to Example 2. Purification was carried as described hereinafter.

Antibody producing cell strains (genetically recombinant CHO-DG44 cells) were cultured in IS CHO-CD/w medium (Irvine Scientific, Cat. No. 91119) containing Hydrolysate supplemented with 4 nM GlutaMax (Invitrogen, Cat 35050-061) and 200 μg/ml of G418 (Sigma, Cat. No. A1720-5G) in a CO₂ incubator under an atmosphere of 5% CO₂ at 37° C. The cells were passaged twice per week. Cell culture solution approximately two weeks after subculturing was centrifuged at 3,500 rpm for five minutes at room temperature. The supernatant was recovered, filtered using a 0.45 μm syringe filter and equilibrated using 50 nM Tris-HCl, pH 7.0. After adding culture medium supernatant to a Hi Trap Protein A HP column (GE Healthcare, Cat No. 17-0402-01), washing was performed using 50 nM Tris-HCl, pH 7.0. Elutions were obtained using 0.1 M citric acid pH 4.0. 400 μl was collected on each occasion and neutralized with 40 μl (or a 10/1 amount) of 1 M Tris-HCl, pH 9.0. After dialyzing twice against a 100 times amount of PBS for 2.5 hours using a Slyde-A-Lyzer 10K Dialysis Cassettes (PIERCE Cat No. 66453), dialysis was performed for 15 to 18 hours on one occasion.

After dialysis, the sample was concentrated using a VIVASPIN 50,000 MWCO PES (VIVASCIENCE Cat No. VS0231). The sample was added to a HiLoad 16/60 superdex 200 prep grade column (GE Healthcare Cat No. 17-1069-01) equilibrated with PBS. The sample was filtered using a 0.22 μm syringe filter and concentrated using a VIVASPIN 50,000 MWCO PES (VIVASCIENCE Cat No. VS0231). The antibody concentration was calculated from an A280 value using a BECKMAN COULTER DU530.

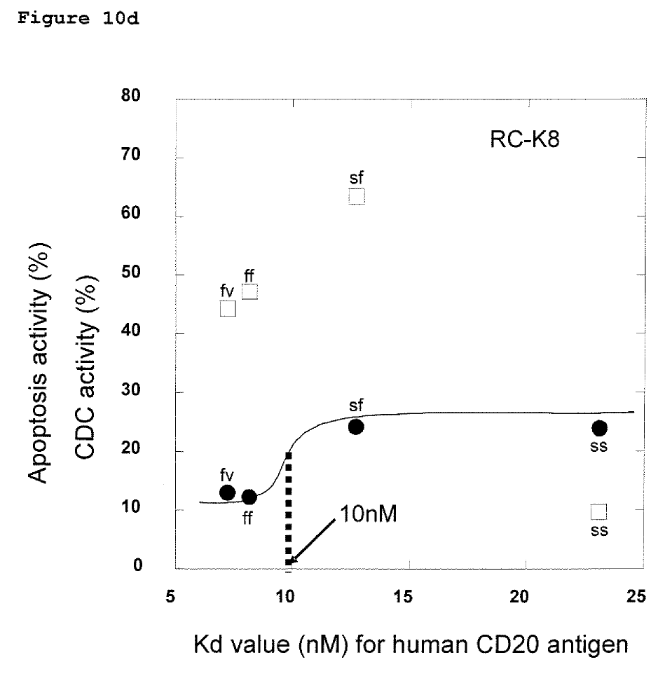

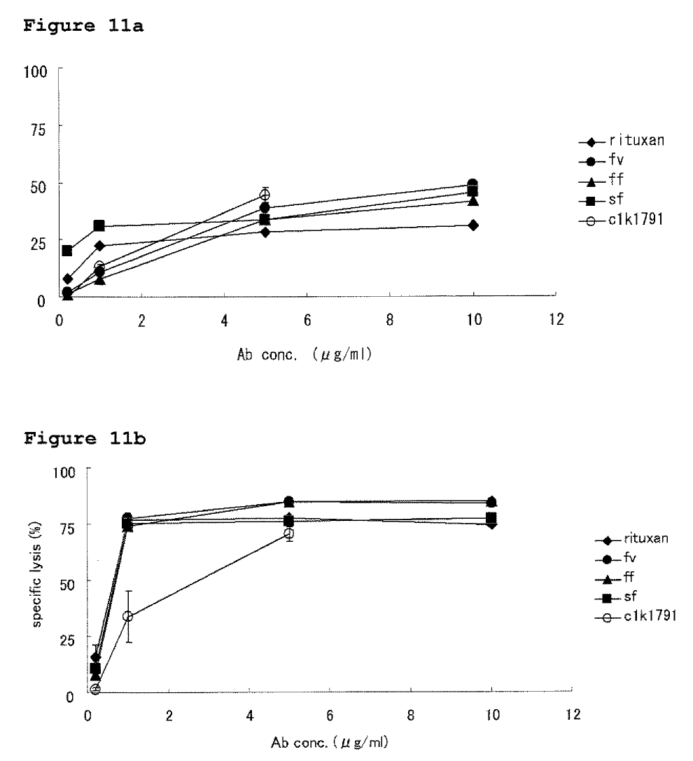

The relationship between concentration and CDC activity of the chimera antibody c1k179 and the humanized antibodies fv, ft, sf, ss against Raji cells, SU-DHL4 cells, WiL-2 cells and RCK8 cells is shown in FIG. 11a to FIG. 11d. In all series of experiments, the CDC activity of the humanized antibodies fv, ff, sf, ss at a concentration of 5 μg/ml or more was equal to or greater than the activity of rituximab (C2B8).

The clones fv and ff (clones expected to display CDC activity) were selected as having a Kd value for Raji cells (floating cells) of less than 9.5 nM and moreover as being antibodies having high CDC activity. These clones displayed higher CDC activity than rituximab with respect to all cell types at a concentration of 5 μg/ml or more. CDC activity with respect to SU-DHL4 cells was particularly high and CDC activity was also high with respect to Raji cells and RCKS cells.

The clone sf (both CDC activity and apoptosis expected) was selected as an antibody which has a Kd value for Raji cells (floating cells) of approximately 9.5 nM to approximately 13 nM, has a total of apoptosis activity and CDC activity as high as possible and has a small Kd value.

This clone displayed cell lytic activity which was equal to or higher than rituximab with respect to all cell types at a concentration of 5 μg/ml or more. Cell lytic activity with respect to SU-DHL4 cells was particularly high and cell lytic activity was also high with respect to WiL2 cells and RCK8 cells.

Apoptosis activity of fv, ff, sf, and ss against Raji cells, SU-DHL4 cells, WiL-2 cells and RCK8 cells was examined and the results are shown in Table 7.

Apoptosis Activity of Humanized Antibodies according to the Present Invention

TABLE 7

| | Affinity | Apoptosis (5 ug/ml) | | | | | | | |
|---|---|---|---|---|---|---|---|---|---|
| | (average) | RAJI | | Wil2-NS | | DHL4 | | RCK8 | |
| | Raji | | XL | | XL | | XL | | XL |
| Antibody | Kd (nM) | m2B8 = 100 | w/wo | m2B8 = 100 | w/wo | m2B8 = 100 | w/wo | m2B8 = 100 | w/wo |
| c2B8 | 5.35 | 100 | 1.4 | 100 | 1.4 | 100 | 1.3 | 100 | 0.9 |
| fv | 7.21 | 88 | 1.9 | 49 | 3.1 | 65 | 2.7 | 50 | 1.9 |
| ff | 8.15 | 84 | 2.2 | 49 | 2.8 | 96 | 1.2 | 47 | 1.9 |
| sf | 12.70 | 205 | 1.0 | 100 | 1.5 | 143 | 1.0 | 93 | 1.2 |
| ss | 23.09 | 202 | 1.1 | 108 | 1.3 | 133 | 1.1 | 92 | 1.2 |
| no Ab | | 42 | 0.9 | 41 | 0.9 | 26 | 2.0 | 37 | 1.1 |

From the results in Table 7, the clone sf has apoptosis inducing activity which is equal to or stronger than rituximab. Sufficient apoptosis activity was induced independently without the need for a secondary antibody. Furthermore the sum of CDC activity and apoptosis activity of the clone sf exceeded that of rituximab with respect to all cells used in the experiment. In particular, a high value was observed with respect to WiL2 cells and SU-DHL4 cells (Table 6, FIG. 11a to FIG. 11d and Table 7).

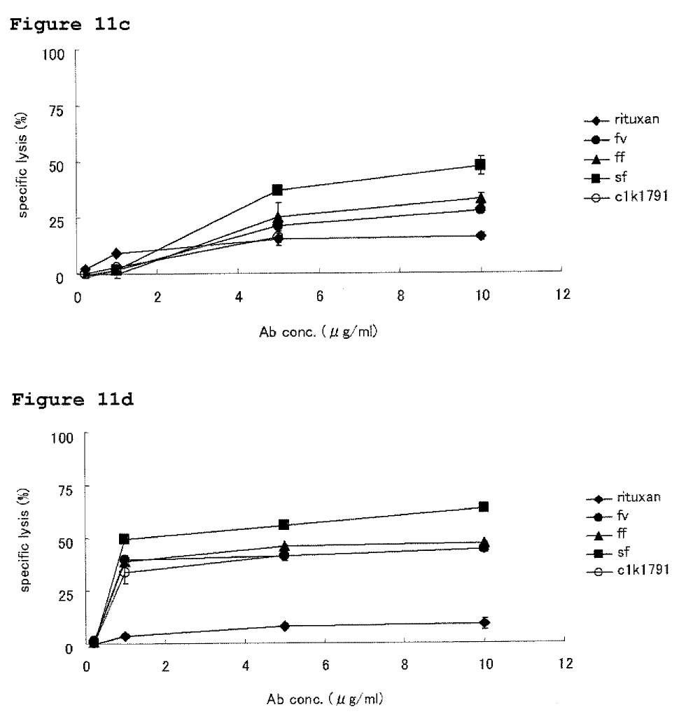

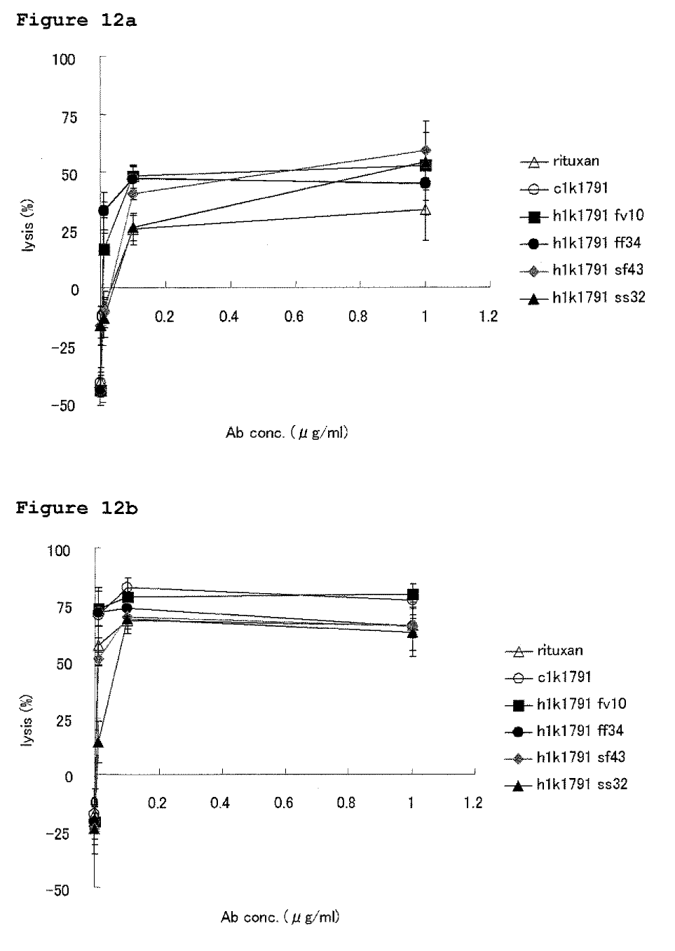

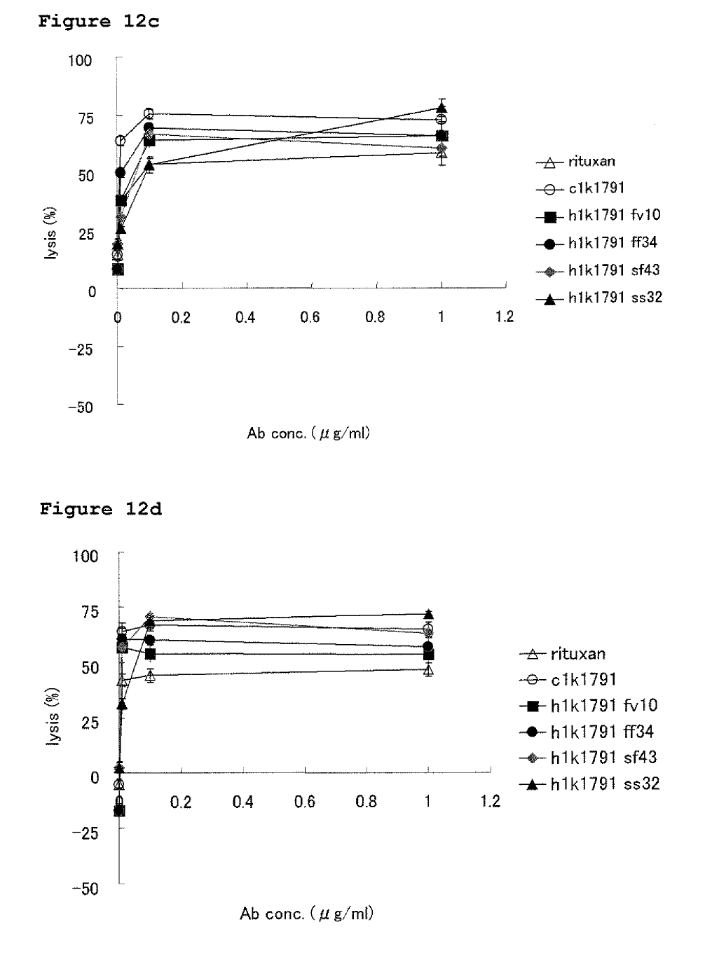

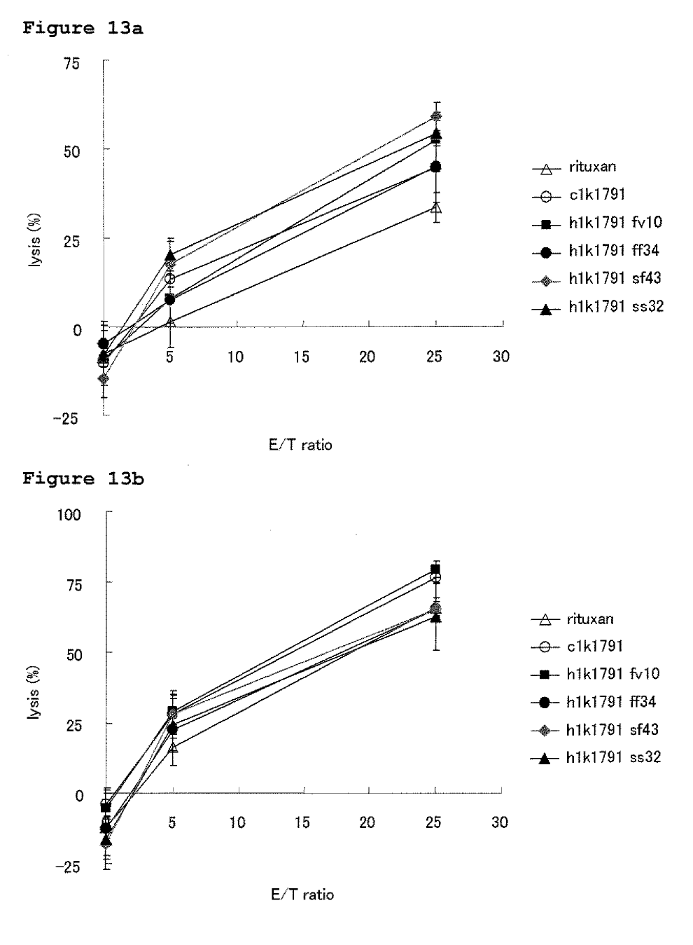

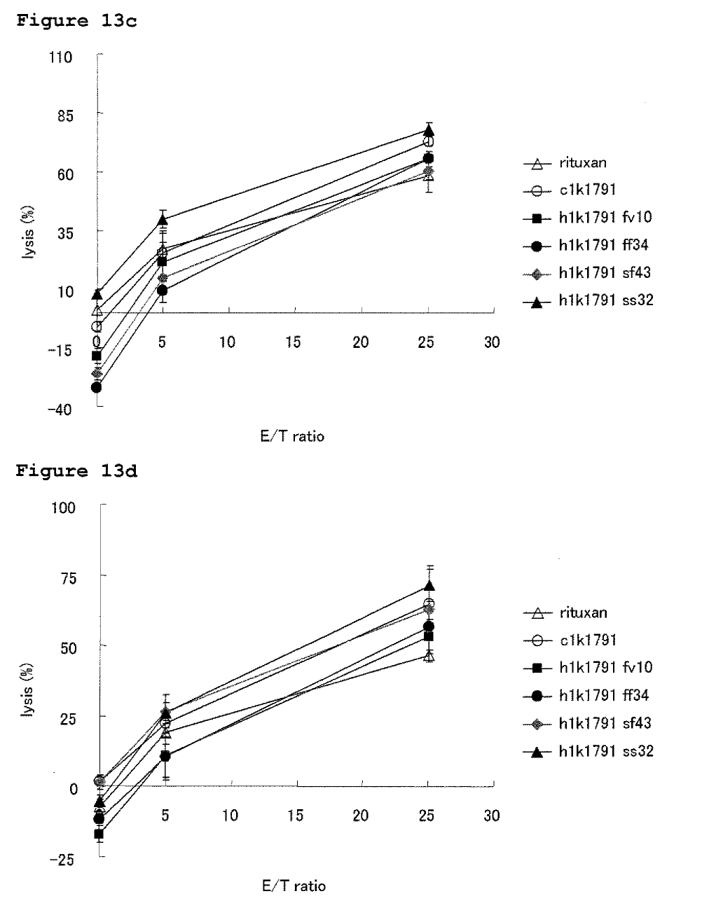

The ADCC activity of fv, ff, sf, and ss against Raji cells, SU-DHL4 cells, WiL-2 cells and RCK8 cells was examined. The relationship between antibody concentration and ADCC is shown in FIG. 12a to FIG. 12d (E:T ratio is 25). The relationship between E:T ratio and ADCC is shown in FIG. 13a to FIG. 13d (antibody concentration is 1 μg/ml). The ADCC activity of fv, ff, sf and ss in all experiments was equal to or greater than the activity of rituximab (C2B8). The results for ADCC activity also demonstrate the efficacy fv, ff, sf and ss selected in this experiment.

These results show that humanized monoclonal antibodies according to the present invention selected in the manner described above display higher cell lytic activity than the cell lytic activity of rituximab. Thus humanized antibodies selected according to the selection criteria of the present invention are thought to have therapeutic efficacy in B cell mediated diseases allowing for their use as pharmaceuticals.

### INDUSTRIAL APPLICABILITY

According to the present invention, a humanized anti human CD20 monoclonal antibody and a method of selection therefor is provided, the antibody displaying biological activity suitable for use as a pharmaceutical and high binding affinity to natural human CD20 molecules. These antibodies have therapeutic efficacy with respect to B cell mediated diseases allowing for their use as pharmaceuticals.

SEQ. ID. NO.: 17: L chain V region sequence of humanized antibody abb 1791
SEQ. ID. NO.: 18: L chain V region sequence of humanized antibody fra 1791
SEQ. ID. NO.: 19: L chain V region sequence of humanized antibody sdr 1791
SEQ. ID. NO.: 20: L chain V region sequence of humanized antibody Ven 1791
SEQ. ID. NO.: 21: H chain V region sequence of humanized antibody abb 1791
SEQ. ID. NO.: 22: H chain V region sequence of humanized antibody fra 1791
SEQ. ID. NO.: 23: H chain V region sequence of humanized antibody sdr 1791
SEQ. ID. NO.: 24: H chain V region sequence of humanized antibody Ven 1791
SEQ. ID. NO.: 25: primer
SEQ. ID. NO.: 26: primer

1. A humanized anti CD20 monoclonal antibody that has growth inhibiting activity on cells containing human CD20 antigen,
wherein said antibody is derived using an immunogen comprising a human B cell strain expressing a human CD20 antigen and a non-human cell strain transformed with human CD20-expressing DNA which non-human cell is derived from an animal which is different from the animal to be immunized, and wherein:
(i) the antibody has a dissociation constant (Kd value) for human CD20 antigen of less than approximately 9.5 nM and CDC activity on B cells equal to or greater than that of 2B8 antibody.

2. A humanized anti CD20 monoclonal antibody that has growth inhibiting activity on cells containing human CD20 antigen,
wherein said antibody is derived using an immunogen comprising a human B cell strain expressing a human CD20 antigen and a non-human cell strain transformed with human CD20-expressing DNA which non-human cell is derived from an animal which is different from the animal to be immunized, and wherein:
(a) the antibody has a dissociation constant (Kd value) for human CD20 antigen of less than approximately 9.5 nM and CDC activity on Raji cells (suspended cells) or SU-DHL4 cells equal to or greater than that of 2B8 antibody.

3. A humanized anti CD20 monoclonal antibody that has growth inhibiting activity on cells containing human CD20 antigen,
wherein said antibody is derived using an immunogen comprising a human B cell strain expressing a human CD20 antigen and a non-human cell strain transformed with human CD20-expressing DNA which non-human cell is derived from an animal which is different from the animal to be immunized, and wherein:
(ii) the antibody has a Kd value for human CD20 antigen in the range of from approximately 9.5 nM to approximately 13 nM and a total of apoptosis activity and CDC activity on B cells equal to or greater than that of 2B8 antibody.

4. A humanized anti CD20 monoclonal antibody that has growth inhibiting activity on cells containing human CD20 antigen,
wherein said antibody is derived using an immunogen comprising a human B cell strain expressing a human CD20 antigen and a non-human cell strain transformed with human CD20-expressing DNA which non-human cell is derived from an animal which is different from the animal to be immunized, and wherein:
(b) the antibody has a Kd value for human CD20 antigen in the range of from approximately 9.5 nM to approximately 13 nM and a total of apoptosis activity and CDC activity on WiL2 cells or RCK8 cells equal to or greater than that of 2B8 antibody.

5. The humanized anti CD20 monoclonal antibody according to claim 1, comprising a combination of the L chain set forth in SEQ ID NO: 18 and the H chain set forth in SEQ ID NO: 22.

6. The humanized anti CD20 monoclonal antibody according to claim 1, comprising a combination of the L chain set forth in SEQ ID NO: 18 and the H chain set forth in SEQ ID NO: 24.

7. The humanized anti CD20 monoclonal antibody according to claim 3, comprising a combination of the L chain set forth in SEQ ID NO: 19 and the H chain set forth in SEQ ID NO: 22.

8. A therapeutic agent for the treatment of B cell mediated diseases, comprising as an active ingredient the humanized anti CD20 monoclonal antibody according to claim 1.

9. The humanized anti CD20 monoclonal antibody according to claim 2, comprising a combination of the L chain set forth in SEQ ID NO: 18 and the H chain set forth in SEQ ID NO: 22.

10. The humanized anti CD20 monoclonal antibody according to claim 2, comprising a combination of the L chain set forth in SEQ ID NO: 18 and the H chain set forth in SEQ ID NO: 24.

11. The humanized anti CD20 monoclonal antibody according to claim 4, comprising a combination of the L chain set forth in SEQ ID NO: 19 and the H chain set forth in SEQ ID NO: 22.
